(19) United States
(12) Patent Application Publication
(10) Pub. No.: US 2023/0005900 A1
(43) Pub. Date: Jan. 5, 2023

(54) CHIP PACKAGE STRUCTURE AND APPLICATION THEREOF

(71) Applicant: iReach Corporation, Hsinchu (TW)

(72) Inventors: Hsiu-Ju YANG, Hsinchu (TW); Hsin-Chan CHUNG, Hsinchu (TW); Shou-Lung CHEN, Hsinchu (TW); Chi-Hsun HSIEH, Hsinchu (TW)

(21) Appl. No.: 17/855,015

(22) Filed: Jun. 30, 2022

Related U.S. Application Data
(60) Provisional application No. 63/216,939, filed on Jun. 30, 2021.

(51) Int. Cl. H01L 25/16; A61N 1/39; H01L 33/62; H01L 31/02; H01L 31/0203; H01L 31/048; H01L 33/48; H01L 31/024; H01L 31/052; H01L 31/0224; H01L 33/64; H01S 5/024; H01S 5/02345; H01S 5/02315; H01S 5/02355

(52) U.S. Cl. CPC H01L 25/167; A61N 1/3904; A61N 1/3975; H01L 33/62; H01L 31/02005; H01L 31/0203; H01L 31/048; H01L 33/483; H01L 31/024; H01L 31/052; H01L 31/022408; H01L 33/641; H01L 33/644; H01S 5/02469; H01S 5/02345; H01S 5/02315; H01S 5/02355

(57) ABSTRACT

A chip package structure includes a substrate having a first surface and a second surface being opposite surfaces of the substrate; a housing disposed on the first surface of the substrate and enclosing a chip region; and a chip set disposed in the chip region and electrically connected to the substrate. The chip set includes a first chip and a second chip, and an active surface of the second chip faces the active surface of the first chip.

### CROSS-REFERENCE TO RELATED APPLICATION

This application claims priority of U.S. provisional application Ser. No. 63/216,939 filed on 30 Jun. 2021, which is incorporated by reference in its entirety.

### TECHNICAL FIELD

The disclosure is related to a chip package structure.

### BACKGROUND

In a chip package structure known to the inventor, an emitter chip and a receiver chip are usually disposed side by side on a substrate; that is, the emitting end and the receiving end of the package structure are on the same plane to form a sensor, and this configuration is adopted in proximity sensor applications. This type of application is commonly installed in mobile devices so that the mobile device is woken up when a target appears in the detection range. Alternatively, during phone calls, when a target appears in the detection range, the sensor can detect the target so that the touch function of the mobile device can be disabled temporarily so as to avoid accidental touch events.

### SUMMARY

In one or some embodiments of the disclosure, a chip package structure is provided and comprises a chip set having an upper chip and a lower chip opposite to each other; that is, according to one or some embodiments, in the chip package structure, one of the chips of the chip set (such as a first chip) is adapted to be electrically driven to emit light as an emitting end, and the other one of the chips of the chip set (such as a second chip) is adapted to be a receiving end to receive light. The emitting end of the first chip and the receiving end of the second chip are configured to be opposite to each other to form a converter which converts optical energy into electrical energy and provides electrical power output.

In one or some embodiments, a converter is also provided and comprises a plurality of the chip package structures. The chip package structures are electrically connected in series so as to realize the objects of high output voltage feature and miniaturized structure of the converter.

In one or some embodiments, a converter is also provided and comprises a plurality of the chip package structures. The chip package structures are electrically connected in parallel so as to realize the objects of large current feature and miniaturized structure of the converter.

According to an exemplary embodiment, a chip package structure comprises: a substrate comprising a first surface and a second surface being opposite surfaces of the substrate; a housing disposed on the first surface of the substrate and enclosing a chip region; and a chip set disposed in the chip region and electrically connected to the substrate, wherein the chip set comprises a first chip and a second chip, and an active surface of the second chip faces an active surface of the first chip.

### BRIEF DESCRIPTION OF THE DRAWINGS

The disclosure will become more fully understood from the detailed description given herein below for illustration only, and thus not limitative of the disclosure, wherein:

FIG. 1A and FIG. 1B illustrate a schematic cross-sectional view and a schematic top view of a chip package structure according to an exemplary embodiment, respectively;

FIG. 2A illustrates a schematic cross-sectional view of a chip package structure according to an exemplary embodiment;

FIG. 2B and FIG. 2C illustrate a schematic top view and a schematic cross-sectional view according to an exemplary embodiment, respectively;

FIG. 3 illustrates a schematic cross-sectional view of a chip package structure according to an exemplary embodiment;

FIG. 4 illustrates a schematic cross-sectional view of a chip package structure according to an exemplary embodiment;

FIG. 5 illustrates a schematic cross-sectional view of a chip package structure according to an exemplary embodiment;

FIG. 6 illustrates a schematic cross-sectional view of a chip package structure according to an exemplary embodiment;

FIG. 7 illustrates a schematic cross-sectional view of a chip package structure according to an exemplary embodiment;

FIG. 8 illustrates a schematic cross-sectional view of a chip package structure according to an exemplary embodiment;

FIG. 9A through FIG. 9F illustrate the manufacture process of a chip package structure according to an exemplary embodiment;

FIG. 10 and FIG. 11 illustrate a schematic cross-sectional view and a schematic bottom (electrode side) view of a chip package structure according to an exemplary embodiment;

FIG. 12 illustrates a schematic top view and an enlarged partial top view of a chip package structure according to an exemplary embodiment;

FIG. 13 illustrates a schematic cross-sectional view of the basic epitaxial structure of a second chip of a chip package structure according to an exemplary embodiment;

FIG. 14A through FIG. 14F illustrate schematic cross-sectional views of the structures of the second chip in all manufacture steps of the manufacturing process of the second chip according to an exemplary embodiment;

FIG. 15A through FIG. 15F illustrate schematic top views of the structures of the second chip in all manufacture steps of the manufacturing process of the second chip corresponding to FIG. 14A through FIG. 14F;

FIG. 16 illustrates a schematic top view of the second chip according to an exemplary embodiment;

FIG. 17A illustrates an enlarged partial view of FIG. 16 and is used to illustrate the structure within block X enclosed by dotted line, and FIG. 17B and FIG. 17C illustrate schematic cross-sectional views of FIG. 17A along line B-B′ and line C-C′, respectively;

FIG. 18A illustrates an enlarged partial view of FIG. 16 and is used to illustrate the structure within the block Y enclosed by dotted line, and FIG. 18B illustrates a schematic cross-sectional view of FIG. 18A along line D-D′;

FIG. 19 illustrates a schematic view of the epitaxial structure of a second chip according to an exemplary embodiment;

FIG. 20 illustrates a schematic view of the epitaxial structure of a second chip according to an exemplary embodiment;

FIG. 21A through FIG. 21C illustrate the manufacture process of a second chip according to an exemplary embodiment;

FIG. 22 illustrates a schematic cross-sectional view of a chip package structure according to an exemplary embodiment;

FIG. 23 illustrates a schematic cross-sectional view of a chip package structure according to an exemplary embodiment;

FIG. 24A and FIG. 24B illustrate schematic perspective views showing the shapes of the chip sets of chip package structure according to two exemplary embodiments of the chip package structure, respectively;

FIG. 25A and FIG. 25B illustrate a schematic top view and a schematic cross- sectional view of a chip package structure according to an exemplary embodiment, respectively;

FIG. 25C illustrates a schematic cross-sectional view of a chip package structure according to another exemplary embodiment;

FIG. 26A through FIG. 26C illustrate optical-microscopic photos showing top views of the chip package structure according to an exemplary embodiment;

FIG. 27 illustrates a schematic diagram of an automated external defibrillator (AED) module according to an exemplary embodiment; and

FIG. 28 illustrates a schematic block diagram of a tunable optical transformer device according to an exemplary embodiment.

### DETAILED DESCRIPTION

The following description is to be understood with the aid of the provided figures, and the concept of the disclosure is illustrated using provided exemplary embodiments. In the figures or the description, identical or similar items are denoted using identical or corresponding numbers/symbols. Besides, the figures are for illustrative purposes, wherein the thickness and shape of each layer are not the actual size or ratio of any corresponding element. The terms “first,” “second,” “third,” etc. are to denote and illustrate features corresponding to the exemplary embodiments but are not to differentiate the elements sequentially, hierarchically, spatially, temporally, etc.

In one or some embodiments, a chip package structure is provided and comprises a chip set having an upper chip and a lower chip opposite to each other; that is, according to one or some embodiments, in the chip package structure, an emitting end and a receiving end are configured to be opposite to each other, and thus a converter is formed. In the converter, one of the chips of the chip set (such as a first chip) is adapted to be an emitting end to emit light/optical radiation, the other one of the chips of the chip set (such as a second chip) is adapted to be a receiving end to receive light/optical radiation, and thus optical energy can be converted into electrical energy for electrical power output.

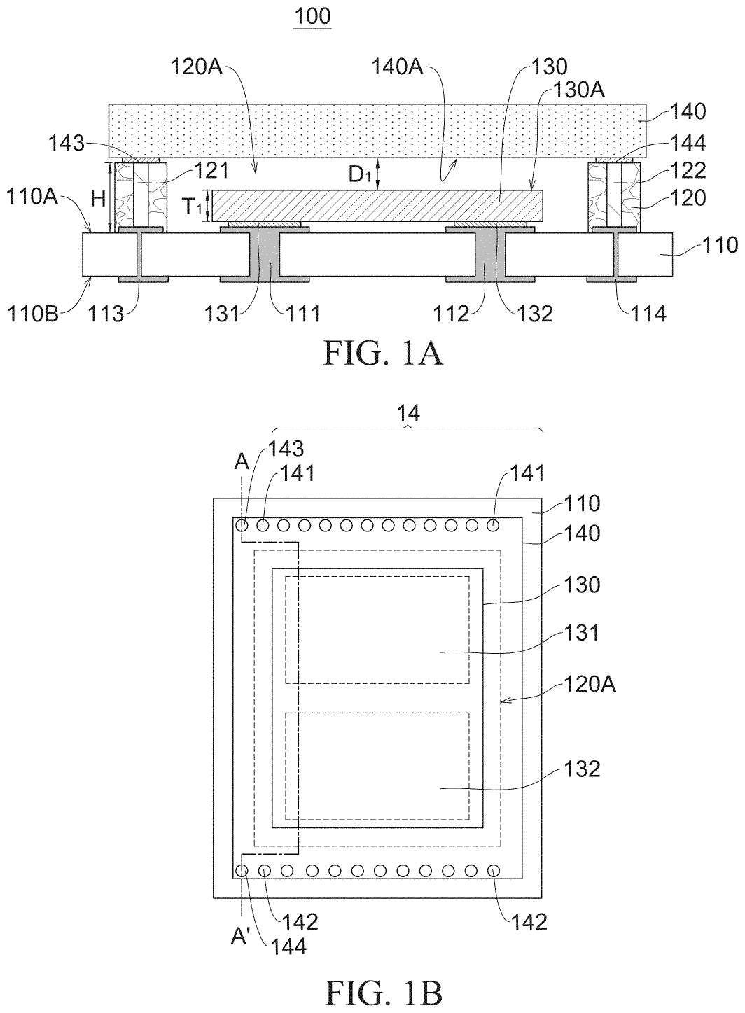

Please refer to FIG. 1A and FIG. 1B. FIG. 1A and FIG. 1B illustrate a schematic cross-sectional view and a schematic top view of a chip package structure according to an exemplary embodiment, respectively, wherein Fig. 1A is the schematic cross-sectional view of FIG. 1B along line A-A′. As shown in FIG. 1A, the chip package structure 100 comprises a substrate 110 and a housing 120 disposed on a first surface 110A of the substrate 110. The substrate 110 is adapted to be the main supportive structure of the chip package structure 100. The chip package structure 100 further comprises a plurality of electrical conduction posts 111, 112, 113, 114. The electrical conduction posts 111, 112, 113, 114 penetrate the substrate 110 and extend to a second surface 110B of the substrate 110, so that the chip package structure 100 can be electrically connected to the exterior through the electrical conduction posts 111, 112, 113, 114. The housing 120 encloses a chip region 120A in the first surface 110A of the substrate 110. The chip package structure 100 comprises a chip set disposed in the chip region 120A and electrically connected to the substrate 110. The chip set comprises a first chip 130 and a second chip 140 opposite to each other, and an active surface 140A of the second chip 140 faces an active surface 130A of the first chip 130.

In this exemplary embodiment, the housing 120 is an electrically conductive housing. The chip package structure 100 further comprises a plurality of first electrical conduction structures 121 and a plurality of second electrical conduction structures 122. The first electrical conduction structures 121 and the second electrical conduction structures 122 penetrate the housing 120 and enclose the first chip 130 disposed on the first surface 110A. The height H of the housing 120 is greater than the thickness T1 of the first chip 130. The second chip 140 is disposed on the housing 120 and electrically connected to the substrate 110 through the first electrical conduction structures 121 and the second electrical conduction structures 122. Specifically, as previously illustrated, in this embodiment, the electrical conduction posts 111, 112, 113, 114 penetrate the substrate 110 and extend to the second surface 110B of the substrate 110. The electrical conduction posts 111, 112 are connected to a first conduction structure 131 and a second conduction structure 132 of the first chip 130, respectively, so that the first chip 130 can be electrically connected to a circuit (not shown in the figures) exterior to the chip package structure 100. The electrical conduction posts 113, 114 are connected to the first electrical conduction structures 121 and the second electrical conduction structures 122 in the housing 120, respectively, so that the second chip 140 on the housing 120 can be electrically connected to a circuit (not shown in the figures) exterior to the chip package structure 100 through the first electrical conduction structures 121, the second electrical conduction structures 122, and the electrical conduction posts 113, 114.

Please also refer to FIG. 1B. The second chip 140 further comprises a connection layer 14. The connection layer 14 comprises a plurality of first aligning connection structures 141, a plurality of second aligning connection structures 142, a first conductive connection structure 143, and a second conductive connection structure 144. The second chip 140 is connected to the first electrical conduction structures 121 and the second electrical conduction structures 122 in the housing 120 through the first conductive connection structure 143 and the second conductive connection structure 144, respectively. The first electrical conduction structures 121 and the second electrical conduction structures 122 are further connected to the electrical conduction posts 113, 114, so that the electrical connection of the second chip 140 to a circuit exterior to the chip package structure 100 can be established. The first aligning connection structures 141 and the second aligning connection structures 142 of the connection layer 14 do not provide electrical connection and are aligned with the first conductive connection structure 143 and the second conductive connection structure 144, respectively, so that the first aligning connection structures 141 and the second aligning connection structures 142 are provided for positioning the second chip 140 in the chip package structure 100. The first aligning connection structures 141 and second aligning connection structures 142 are configured to be asymmetrical, so that a grip force formed due to the aligning connection structures on one side of the chip is different from the grip force formed due to the aligning connection structures on the other side of the chip, so as to prevent the chip from moving during alignment. The aforementioned asymmetrical configuration may refer to that the number of the first aligning connection structures 141 and the number of the second aligning connection structures 142 are different, for example, and/or the relative positions among the first aligning connection structures 141 and the relative positions among the second aligning connection structures 142 are different, for example, the distance between two neighboring ones of the first aligning connection structures 141 and the distance between two neighboring ones of the second aligning connection structures 142 are different, but the disclosure is not limit thereto.

In this exemplary embodiment, the first chip 130 is a light-emitting chip, such as a light-emitting diode (LED) or a laser diode, and an active surface of the light-emitting chip is the light-emitting surface; the second chip 140 is a light-receiving chip, such as a photovoltaic chip or a photodiode chip, and an active surface of the light-receiving chip is the light-receiving surface or the light-sensing surface. In this exemplary embodiment, the first chip 130 may be a vertical cavity surface emitting laser (VCSEL) chip or a flip chip laser diode, but the disclosure is not limited thereto. In some other exemplary embodiments, the first chip is a light-receiving chip, and the second chip is a light-emitting chip. In this exemplary embodiment, the emitting surface 130A of the first chip 130 faces the receiving surface 140A of the second chip 140. The receiving surface 140A of the second chip 140 receives light emitted by the first chip 130, and the area of the receiving surface 140A is greater than the area of the emitting surface 130A of the first chip 130. In this exemplary embodiment, a distance D1 exists between the emitting surface 130A of the first chip 130 and the receiving surface 140A of the second chip 140. The length of the distance D1 is determined by the application of the chip package structure 100 and/or photoelectric characteristics of the first chip 130 and photoelectric characteristics of the second chip 140. In this exemplary embodiment, the distance D1 between the emitting surface 130A of the first chip 130 and the receiving surface 140A of the second chip 140 is in a range between 1 μm and 30 μm.

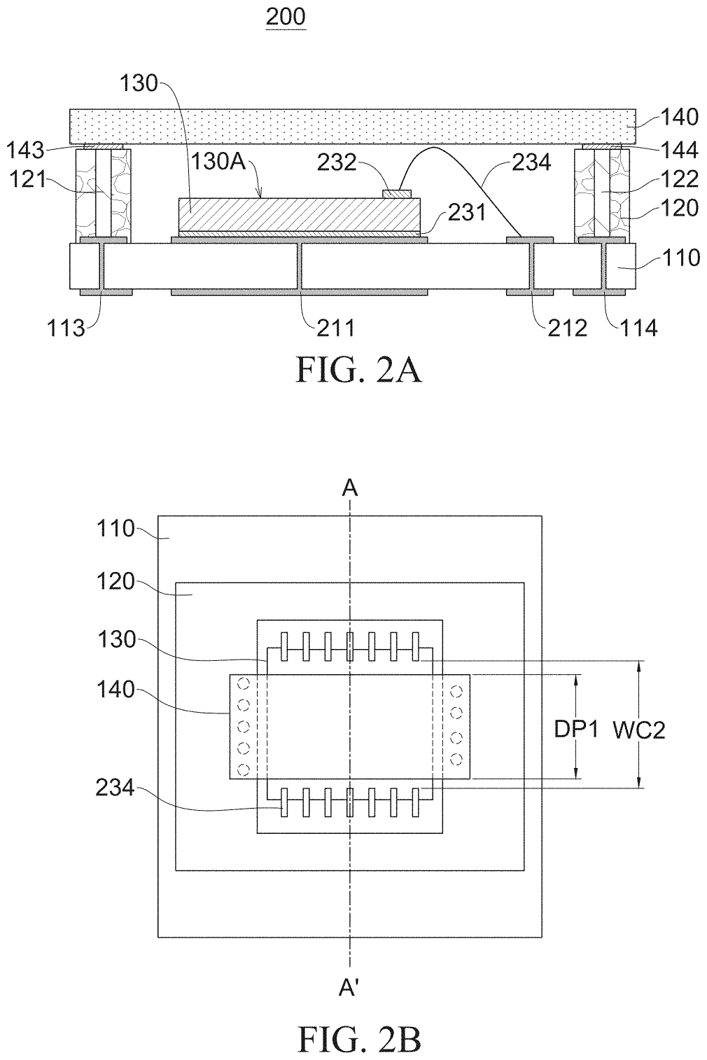

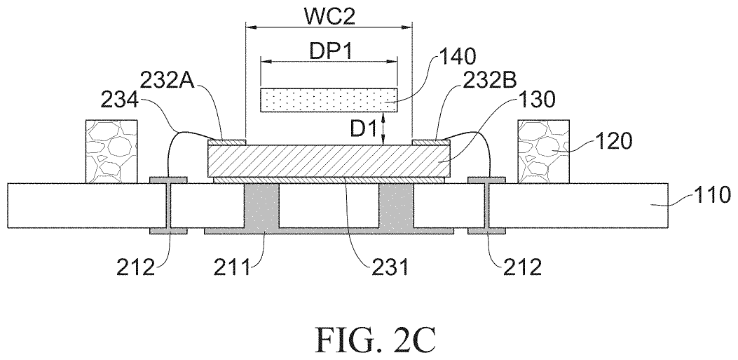

Please refer to FIG. 2A. FIG. 2A illustrates a schematic cross-sectional view of a chip package structure according to an exemplary embodiment. As shown in FIG. 2A, the chip package structure 200 comprises similar elements and structure to those of the chip package structure 100 shown in FIG. 1A, and the difference between the embodiment shown in FIG. 1A and the embodiment shown in FIG. 2A is at least that, in the chip package structure 200, the first chip 130 is a vertical chip. In other words, in this embodiment, the first conduction structure 231 and the second conduction structure 232 are on opposite surfaces of the first chip 130, respectively. The first conduction structure 231 is connected to the first electrical conduction post 211. The second conduction structure 232 disposed on the emitting surface 130A of the first chip 130 is connected to the first electrical conduction post 212 through a wire 234. In this exemplary embodiment, due to the connection between the first conduction structure 231 and the first electrical conduction post 211 plus the connection between the second conduction structure 232 and the first electrical conduction post 212 through the wire 234, the first chip 130 is electrically connected to the substrate 110, so that the electrical conduction between the first chip 130 and the exterior of the chip package structure 100 can be established.

Please refer to FIG. 2B and FIG. 2C. FIG. 2B and FIG. 2C illustrate a schematic top view and a schematic cross-sectional view according to an exemplary embodiment, respectively, wherein FIG. 2C illustrates a schematic cross-sectional view of FIG. 2B along line A-A′. As shown in FIG. 2C, the first chip 130 is a vertical chip. In other words, in this embodiment, the first conduction structure 231 and the second conduction structures 232A, 232B are on opposite surfaces of the first chip 130, respectively. The second conduction structures 232A, 232B are on opposite sides of the active surface 130A of the first chip 130. In this exemplary embodiment, the orientations of the second conduction structures 232A, 232B on the first chip 130 are different from the orientations of the first conductive connection structure 143 and the second conductive connection structure 144 on the second chip 140, so that the height of a metal wire (i.e., the wire 234) for the first chip 130 does not limit the distance D1 between the first chip 130 and the second chip 140.

Please continue referring to FIG. 2C. A distance DP1 exists between the second conduction structure 232A and the second conduction structure 232B on two sides of the first chip 130, and the second chip 140 has a width WC2. Preferably, in one embodiment, the distance DP1 is greater than the width WB2, so that the height of the metal wire (i.e., the wire 234) for the first chip 130 does not limit the distance D1 between the first chip 130 and the second chip 140. Besides, the wire 234 can be kept from contacting the second chip 140 by accident, but the disclosure is not limited thereto.

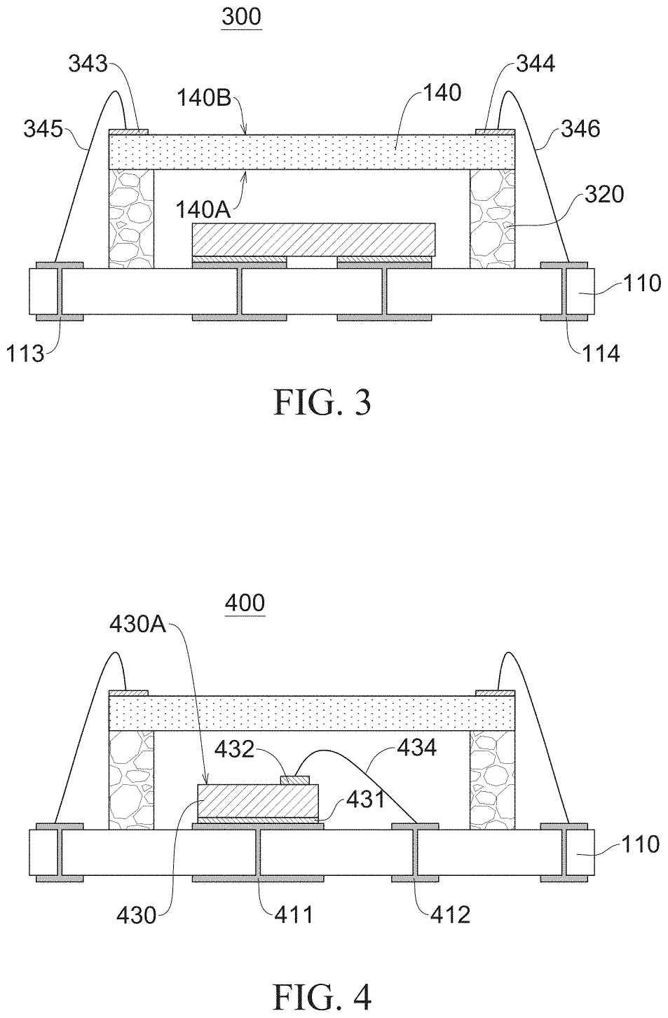

Please refer to FIG. 3. FIG. 3 illustrates a schematic cross-sectional view of a chip package structure according to another exemplary embodiment. As shown in FIG. 3, the chip package structure 300 comprises similar elements and structure to those of the chip package structure 100 shown in FIG. 1A, and the difference between the embodiment shown in FIG. 1A and the embodiment shown in FIG. 3 is at least that, in the chip package structure 300, the housing 320 is not a conductive housing (i.e., there is no electrical conduction structure that penetrates the housing). Instead, the housing 320 is just adapted to be a supportive structure in this exemplary embodiment. In this exemplary embodiment, the second chip 140 is a horizontal chip. That is, in this embodiment, the conductive connection structures 343, 344 are on the same surface. As shown in FIG. 3, the first conductive connection structure 343 and the second conductive connection structure 344 are both on an opposite surface 140B of the second chip 140 opposite to the receiving surface 140A. The first conductive connection structure 343 and the second conductive connection structure 344 are electrically connected to the second electrical conduction posts 113, 114 through the wires 345, 346, respectively. In other words, in this exemplary embodiment, due to the wire 345 connected between the first conductive connection structure 343 and the second electrical conduction post 113 plus the wire 346 connected between the second conductive connection structure 344 and the second electrical conduction post 114, the second chip 140 is electrically connected to the substrate 110, so that the second chip 140 can be electrically connected to the exterior of the chip package structure 300.

Please refer to FIG. 4. FIG. 4 illustrates a schematic cross-sectional view of a chip package structure according to another exemplary embodiment. As shown in FIG. 4, the chip package structure 400 comprises similar elements and structure to those of the chip package structure 300 shown in FIG. 3, and the difference between the embodiment shown in FIG. 3 and the embodiment shown in FIG. 4 is at least that, in the chip package structure 400, the first conduction structure 431 and the second conduction structure 432 are on opposite surfaces of the first chip 430, respectively. The first conduction structure 431 is connected to the first electrical conduction post 411. The second conduction structure 432 is on the emitting surface 430A of the first chip 430 and is connected to the first electrical conduction post 412 through the wire 434. Similar to the exemplary embodiment shown in FIG. 2A, the first chip 430 of the chip package structure 400 is a vertical chip. In this exemplary embodiment, due to the connection between the first conduction structure 431 and the first electrical conduction post 411 plus the connection between the second conduction structure 432 and the first electrical conduction post 412 through the wire 434, the first chip 430 is electrically connected to the substrate 110, so that the first chip 430 can be electrically connected to the exterior of the chip package structure 400.

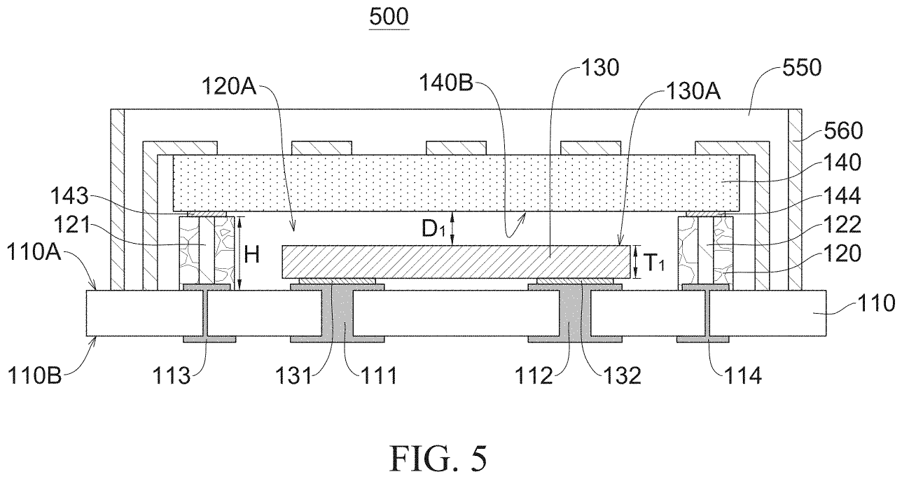

Please refer to FIG. 5. FIG. 5 illustrates a schematic cross-sectional view of a chip package structure according to an exemplary embodiment. As shown in FIG. 5, the chip package structure 500 comprises similar elements and structure to those of the chip package structure 100 shown in FIG. 1A, and the difference between the embodiment shown in FIG. 1A and the embodiment shown in FIG. 5 is at least that the chip package structure 500 further comprises a heat dissipation layer 550. The heat dissipation layer 550 is on the second chip 140 and directly contacts the second chip 140 for heat conduction, so that the heat of the second chip 140 can be dissipated through the dissipation layer 550. Especially, in one or some embodiments, the heat dissipation layer 550 is further connected to the substrate 110 through the periphery of the second chip 140, so as to conduct the heat generated by the second chip 140 to the substrate 110. In this exemplary embodiment, the heat dissipation layer 550 may be a metal layer, such as, but not limited to, silver paste, which has good heat conduction property and is thus beneficial for heat dissipation, and the silver paste can also prevent light leakage of the chip package structure 500. Alternatively, in some embodiments, the heat dissipation layer 550 may also be a plurality of metal layers stacked together, such as, but not limited to, silver paste and electroplated gold, which further protect the heat dissipation layer 550 against problems due to oxidization such as deterioration of heat dissipation effect. In this exemplary embodiment, the heat dissipation layer 550 is positioned by a support structure 560 which is partially on the substrate 110 and partially on the second chip 140. In this exemplary embodiment, the support structure 560 is made of electrically nonconductive material(s), such as, but not limited to, silicon, epoxy resin, plastic material(s), or ceramic material(s).

Please refer to FIG. 5. In an exemplary embodiment, the space between the first chip 130 and the second chip 140 or the space surrounding the first chip 130 may be filled with air, but the disclosure is not limited thereto. In another exemplary embodiment, the space between the first chip 130 and the second chip 140 may be filled with an electrically insulating light-transmissive gel, such as, but not limited to, bisbenzocyclobutene (BCB) gel. Therefore, the difference between a refractive index Nc1 (the refractive index of the structure within the first chip 130 closest to the light-emitting surface, not shown in the figures) and a refractive index Ne (the refractive index Ne is the refractive index of the material in the periphery of the first chip 130, not shown in the figures) can be reduced. Hence, the deflection of the light emitting angle can be reduced, and thus the overall efficiency of photoelectric conversion of the chip package structure 500 can be increased. Moreover, the electrically insulating gel also provides the chip package structure 500 with electrical protection to keep electric arcs caused by high voltage from influencing the chip package structure 500. For example, when the output voltage of at least one of the first chip 130 and the second chip 140 is a high voltage (such as the case for a photovoltaic chip), electric arcs can easily occur and thus influence the operation of the chip or the package. For this reason, the electrically insulating gel filled between the first chip 130 and the second chip 140 can keep electric arcs from happening.

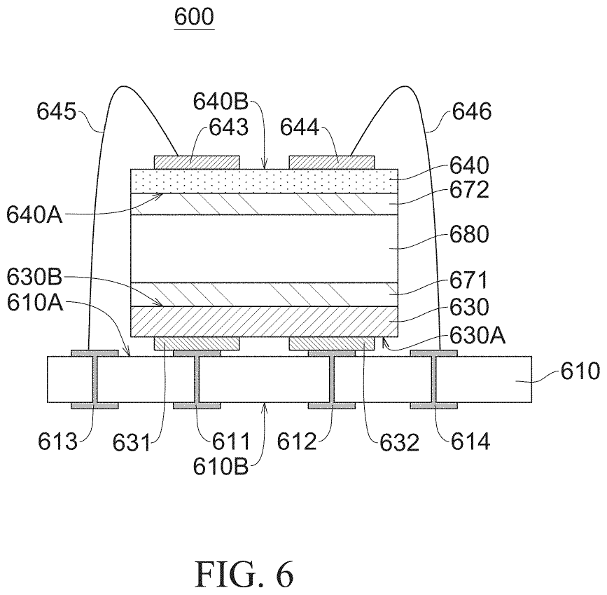

Please refer to FIG. 6. FIG. 6 illustrates a schematic cross-sectional view of a chip package structure according to another exemplary embodiment. In this exemplary embodiment, the chip package structure 600 is a wafer-level packaging converter; that is, in this embodiment, the first chip 630 and the second chip 640 of the chip package structure 600 are combined using a wafer-to-wafer bonding process.

As shown in FIG. 6, in this exemplary embodiment, the chip package structure 600 comprises a substrate 610. The substrate 610 comprises a first surface 610A and a second surface 610B being opposite surfaces of the substrate 610. The chip package structure 600 further comprises at least two first electrical conduction posts 611, 612 and at least two second electrical conduction posts 613, 614. The first electrical conduction posts 611, 612 and the second electrical conduction posts 613, 614 penetrate the substrate 610. The chip package structure 600 also comprises a chip set having a first chip 630 and a second chip 640. In this exemplary embodiment, the first chip 630 and the second chip 640 are connected to a light-transmissive middle layer 680 through a first bonding layer 671 and a second bonding layer 672, respectively. The receiving surface 640A of the second chip 640 faces the emitting surface 630B of the first chip 630 so as to receive the light emitted by the first chip 630. In this exemplary embodiment, the first electrical conduction posts 611, 612 can connect the first chip 630 to the exterior of the converter (the chip package structure 600), and the second electrical conduction posts 613, 614 can connect the second chip 640 to the exterior of the converter (the chip package structure 600).

Specifically, in this exemplary embodiment, the first chip 630 comprises a third surface 630A and a fourth surface 630B. The third surface 630A faces the first surface 610A, the fourth surface 630B faces away the first surface 610A, and the third surface 630A and the fourth surface 630B are opposite surfaces of the first chip 630. The second chip 640 comprises a fifth surface 640A and a sixth surface 640B. The fifth surface 640A faces the fourth surface 630B, the sixth surface 640B faces away the fourth surface 630B, and the fifth surface 640A and the sixth surface 640B are opposite surfaces of the second chip 640. The fourth surface 630B is the emitting surface of the first chip 630, and the fifth surface 640A is the receiving surface of the second chip 640. In this exemplary embodiment, the chip package structure 600 further comprises a first conduction structure 631 and a second conduction structure 632. The first conduction structure 631 and the second conduction structure 632 are on the third surface 630A of the first chip 630 and respectively connected to the first electrical conduction posts 611, 612, so that the first chip 630 is electrically connected to the first electrical conduction posts 611, 612. As a result, the first chip 630 can be electrically connected to the exterior of the converter (the chip package structure 600). The chip package structure 600 further comprises a first conductive connection structure 643 and a second conductive connection structure 644. The first conductive connection structure 643 is on the sixth surface 640B and connected to the second electrical conduction post 613 through a first wire 645, and the second conductive connection structure 644 is on the sixth surface 640B and connected to the second electrical conduction post 614 through a second wire 646, so that the second chip 640 is electrically connected to the second electrical conduction posts 613, 614. As a result, the second chip 640 can be connected to the exterior of the converter (the chip package structure 600).

In this exemplary embodiment, the first bonding layer 671, the second bonding layer 672, and/or the light-transmissive middle layer 680 of the chip package structure 600 may be made of electrically insulating material(s). Compared with the chip package structure 100 shown in FIG. 1A, in this embodiment, the distance D1 between the first chip 630 and the second chip 640 can be reduced because of the wafer-to-wafer bonding process, and the overall volume of the chip package structure 600 can be then reduced. As a result, in this embodiment, the chip package structure 600 is beneficial for small-size device applications.

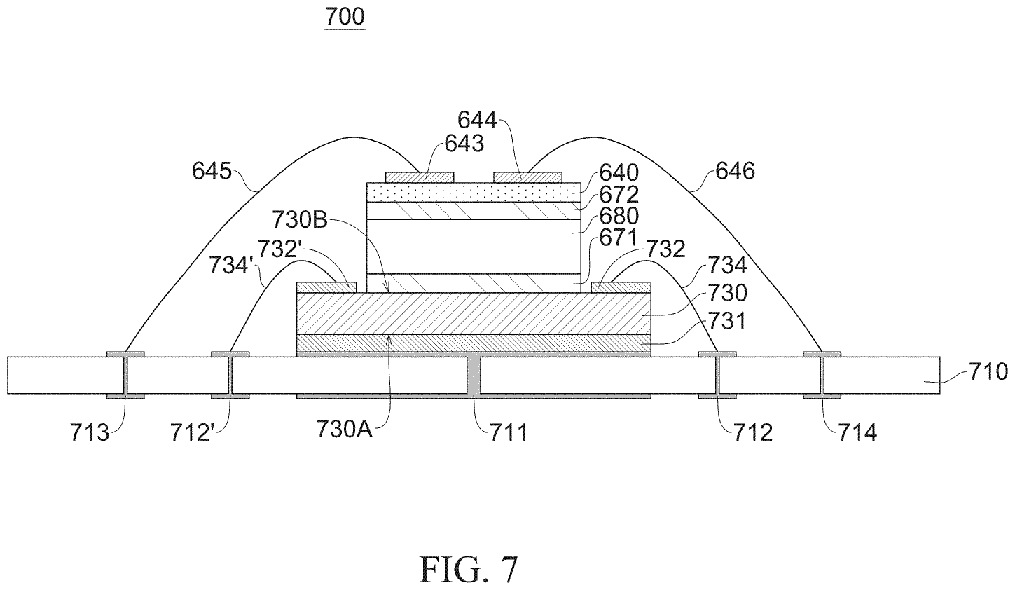

Please refer to FIG. 7. FIG. 7 illustrates a schematic cross-sectional view of a chip package structure according to another exemplary embodiment. As shown in FIG. 7, the chip package structure 700 comprises similar elements and structure to those of the chip package structure 600 shown in FIG. 6, and the difference between the embodiment shown in FIG. 6 and the embodiment shown in FIG. 7 is at least that, in the chip package structure 700, the first chip 730 is a vertical chip rather than the flip chip in the chip package structure 600, and the width of the first chip 730 is greater than the width of the second chip 640.

Specifically, in this embodiment, the chip package structure 700 comprises a first conduction structure 731 and a plurality of second conduction structures 732, 732′. The first conduction structure 731 is on the third surface 730A of the first chip 730, and the second conduction structures 732, 732′ are on the fourth surface 730B of the first chip 730. The first conduction structure 731 is connected to the first electrical conduction post 711, and the second conduction structures 732, 732′ are respectively connected to the first electrical conduction posts 712, 712′ through corresponding wires 734, 734′, so that the first chip 730 is electrically connected to the substrate 710, and thus the first chip 730 can be electrically connected to the exterior of the converter (the chip package structure 700). The formation of the electric conduction between the second chip 640 and the exterior of the converter (the chip package structure 700) is identical to the formation of the electric conduction between the second chip 640 and the exterior of the converter (the chip package structure 600) of the exemplary embodiment shown in FIG. 6.

In this exemplary embodiment, during the manufacture of the chip package structure, a plurality of second chips is bonded to a first wafer (not shown in the figures), wherein the first wafer is formed with a plurality of first chips by cutting the light- transmissive layer. Then, the first wafer is cut. Afterwards, a first conduction structure and a second conduction structure are formed on the obtained structure, and the first chip is disposed on a substrate. In an exemplary embodiment, the first chip and the second chip are electrically connected to the substrate through wires to complete the chip package structure of this embodiment.

As previously described, the second chip is connected to the first chip through a chip-to-wafer bonding process. Similarly, the first chip may also be connected to the second chip through the chip-to-wafer bonding process; that is, in this embodiment, a plurality of first chips is bonded to a second wafer having a plurality of second chips, and then the second wafer is cut. Afterwards, a first conduction structure and a second conduction structure are formed on the obtained structure, and the first conduction structure and the second conduction structure are then disposed on and electrically connected to the substrate to complete the chip package structure of this embodiment, wherein the width of the second chip is greater than the width of the first chip. In an exemplary embodiment, after the packaging process, chips made from epitaxial wafers with higher yields (the first wafer or the second wafer) are closer to the substrate.

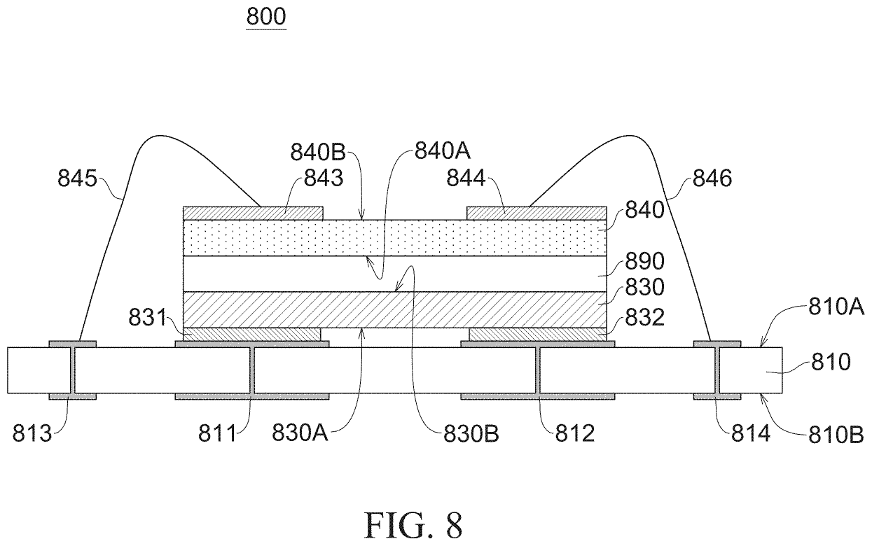

Please refer to FIG. 8. FIG. 8 illustrates a schematic cross-sectional view of a chip package structure according to an exemplary embodiment. In this exemplary embodiment, the first chip 830 and the second chip 840 of the chip package structure 800 are both horizontal chips. The difference between this exemplary embodiment and the previous exemplary embodiments is at least that, in the chip package structure 800, the first chip 830 and the second chip 840 are connected to each other through a spacing layer 890 and electrically insulated from each other by the spacing layer 890. In this exemplary embodiment, the spacing layer 890 may be a light-transmissive layer, such as a light-transmissive middle layer, and the spacing layer 890 may be a single-layered structure or a multi-layered structure, but the disclosure is not limited thereto. In this exemplary embodiment, the electric connection between the first chip 830 and the substrate 810 and the electric connection between the second chip 840 and the substrate 810 are identical to those of the exemplary embodiment shown in FIG. 6; that is, in the chip package structure 800 of this embodiment, The first conduction structure 831 and the second conduction structure 832 are on the third surface 830A of the first chip 830 and respectively connected to the first electrical conduction posts 811, 812, so that the first chip 830 is electrically connected to the first electrical conduction posts 811, 812. As a result, the first chip 830 can be connected to the exterior of the converter (the chip package structure 800). The first conductive connection structure 843 is disposed on the sixth surface 840B and connected to the second electrical conduction post 813 through a first wire 845, and the second conductive connection structure 844 is on the sixth surface 840B and connected to the second electrical conduction post 814 through a second wire 846, so that the second chip 840 is electrically connected to the second electrical conduction posts 813, 814. As a result, the second chip 840 can be connected to the exterior of the converter (the chip package structure 800).

In this exemplary embodiment, the spacing layer and the first chip can be sequentially formed on the second chip by epitaxy. The material of the spacing layer may be a semiconductor with low doping concentration (such as between 1×10¹⁵ cm⁻³ and 5×10¹⁶ cm⁻³) and high resistance. In an exemplary embodiment, the spacing layer can be made of Al₂O₃ obtained by oxidizing AlGaAs.

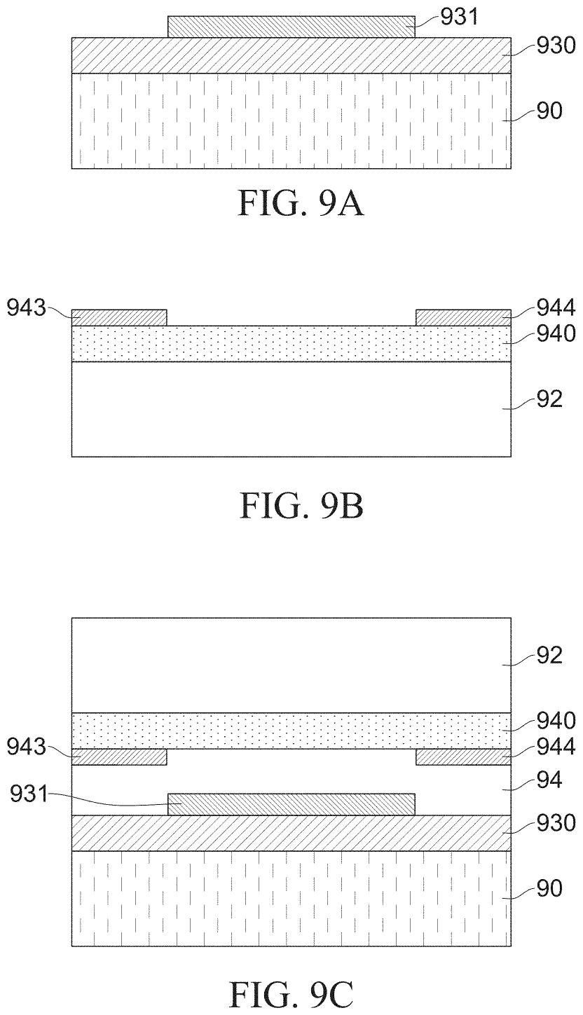

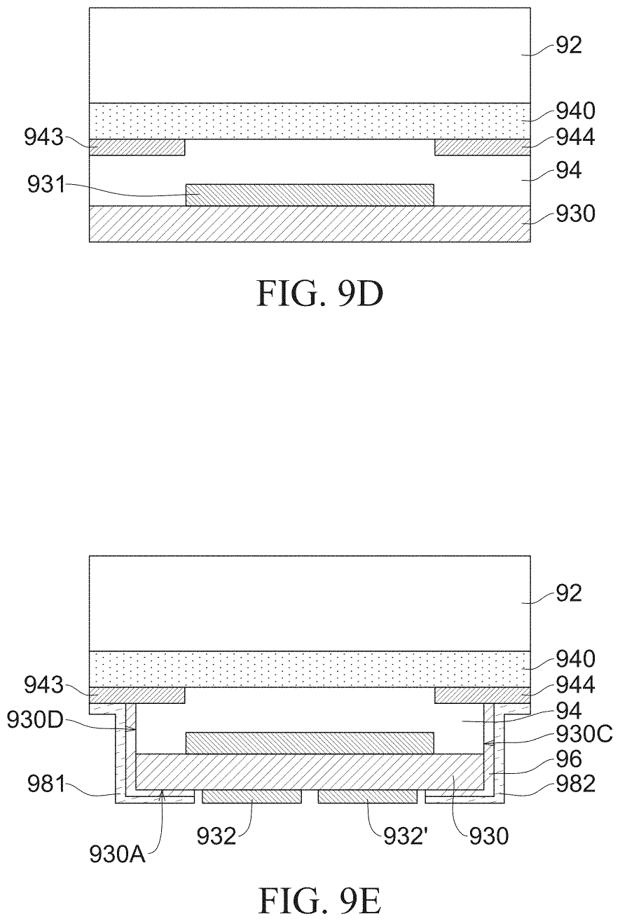

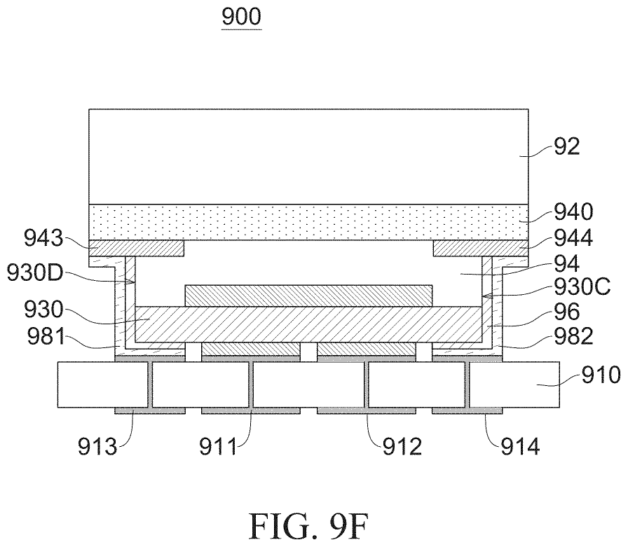

Please refer to FIG. 9A through FIG. 9F. FIG. 9A through FIG. 9F illustrate the manufacture process of a chip package structure according to an exemplary embodiment. In this exemplary embodiment, the chip package structure is a wafer-level package structure formed through the wafer-to-wafer bonding process.

First, as shown in FIG. 9A, a first chip 930 is formed on a growth substrate 90, and a first conduction structure 931 is deposited on the first chip 930. The width of the first conduction structure 931 is less than the width of the first chip 930, so that the first chip 930 is partially exposed.

Next, as shown in FIG. 9B, a second chip 940 is formed on a light-transmissive substrate 92, and a first conductive connection structure 943 and a second conductive connection structure 944 are formed on the same side of the second chip 940.

A bonding layer 94 is used to bond the structure shown in FIG. 9A and the structure shown in FIG. 9B with each other to obtain the structure shown in FIG. 9C.

Then, as shown in FIG. 9D, the growth substrate 90 is removed.

Then, the portions surrounding the first chip 930 and surrounding the bonding layer 94 are removed, so that the first conductive connection structure 943 and the second conductive connection structure 944 are partially exposed.

As shown in FIG. 9E, an insulating layer 96 is formed and covers parts of the surface of the first chip 930 and the side walls 930C, 930D of the first chip 930, and the insulating layer 96 is connected to the first conductive connection structure 943 and the second conductive connection structure 944 through fan-out pads 981, 982, respectively. The fan-out pads 981, 982 also cover the insulating layer 96.

Last, the second conduction structures 932, 932′ are formed on the exposed surface of the first chip 930, and the whole package structure is disposed on the substrate 910 to obtain the chip package structure 900 shown in FIG. 9F, wherein the second conduction structures 932, 932′ are connected to the first electrical conduction posts 911, 912, and the first conductive connection structure 943 and the second conductive connection structure 944 are connected to the second electrical conduction posts 913, 914, respectively, so that the chip package structure can be electrically connected to the exterior.

As previously illustrated, in the previous exemplary embodiments, the first chip may be a light-emitting chip, such as an LED or a laser diode, and the second chip may be a light-receiving chip, such as a photovoltaic chip or a photodiode chip. In the chip package structure, the first chip and the second chip are configured to be opposite to each other in the chip region. The following paragraphs will further illustrate the structures of the first chip and the second chip in the chip package structure according to one or some embodiments.

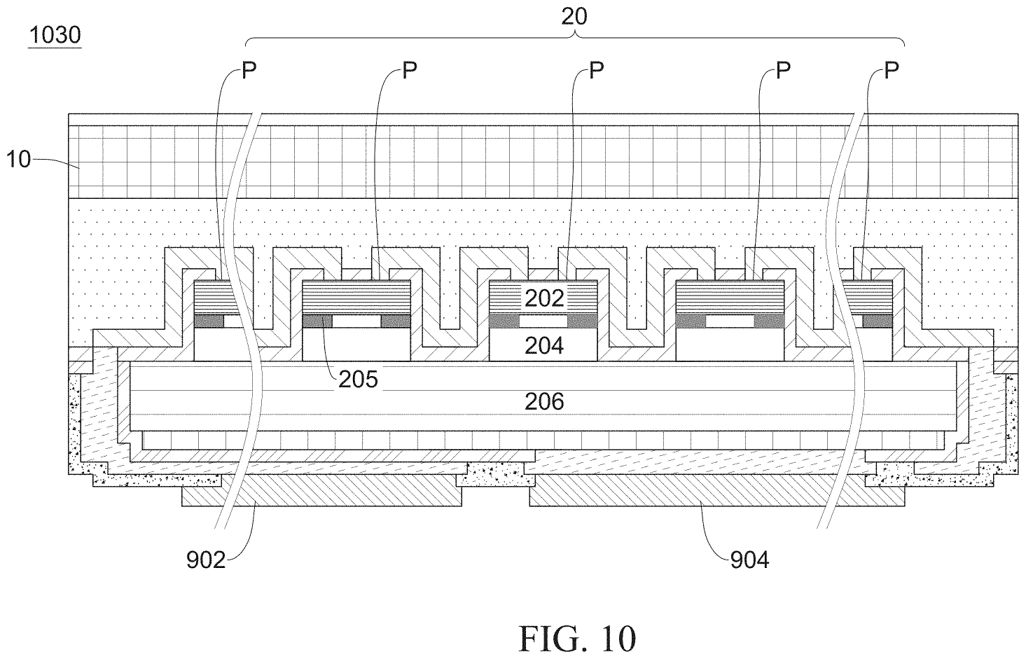

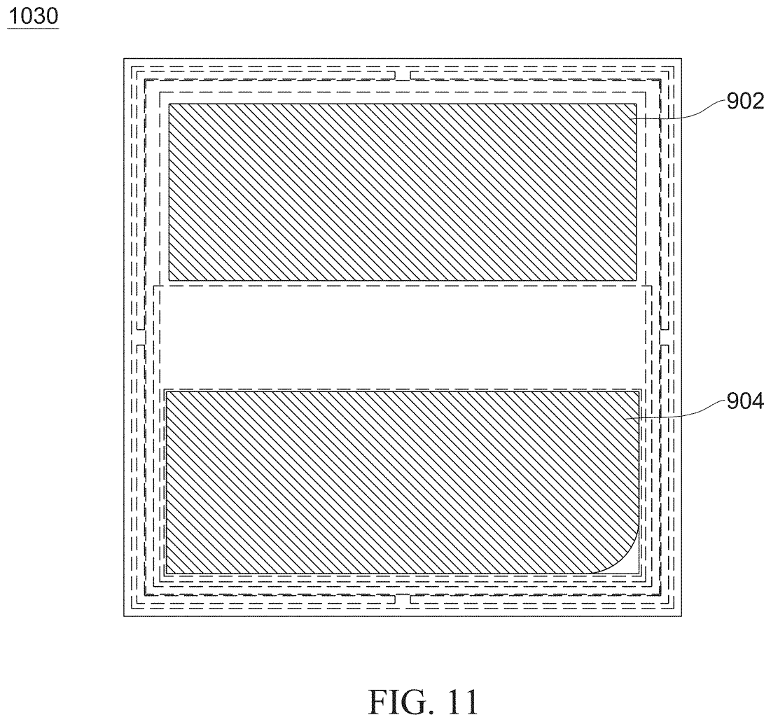

Please refer to FIG. 10 through FIG. 12 for the following illustration on the details of the first chip of the chip package structure according to an exemplary embodiment.

Please refer to FIG. 10 and FIG. 11. FIG. 10 and FIG. 11 illustrate a schematic cross-sectional view and a schematic bottom (electrode side) view of a chip package structure according to an exemplary embodiment. In this exemplary embodiment, the first chip 1030 is a laser element; more specifically, in this embodiment, the first chip 1030 is a VCSEL chip. As shown in FIG. 10, in this exemplary embodiment, the first chip 1030 comprises a permanent substrate 10 and an epitaxy structure 20 on one side of the permanent substrate 10, and the epitaxy structure 20 comprises at least one columnar structure P. In this embodiment, the epitaxy structure 20 comprises a plurality of columnar structures P. Each of the columnar structures P comprises, looking from the permanent substrate 10, a first semiconductor structure 202, a current limiting layer 205, and an active structure 204 in which the first semiconductor structure 202, the current limiting layer 205, and the active structure 204 are sequentially further from the permanent substrate 10. For the specific structure of the first chip 1030 in this exemplary embodiment, please further refer to U.S. application Ser. No. 16/863,277 and Taiwan Patent Application No. 108115024 filed by the applicant.

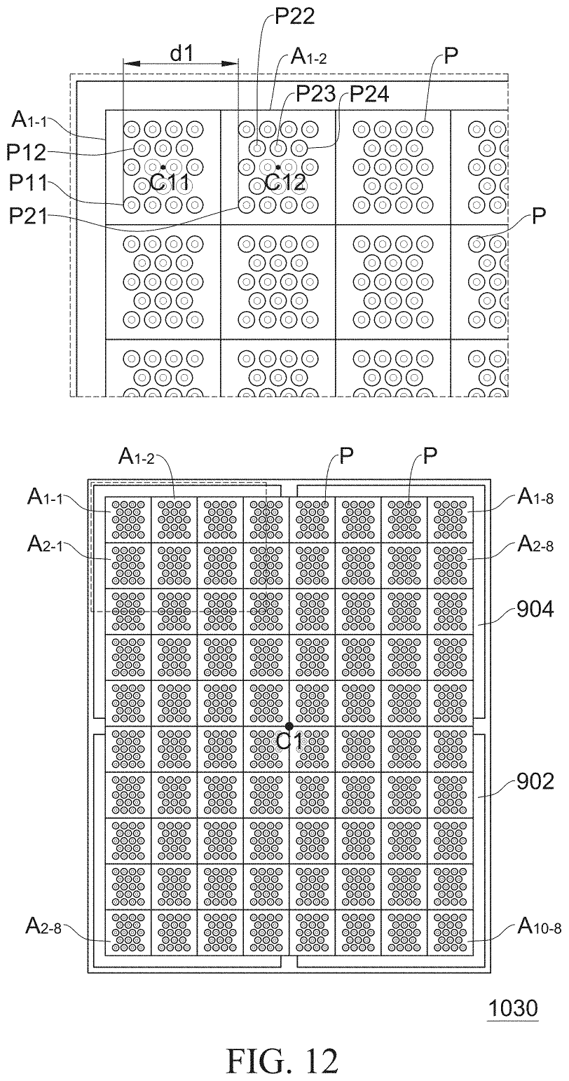

Please refer to FIG. 12. FIG. 12 illustrates a schematic top (light-emitting apertures side) view and an enlarged partial top view of a chip package structure according to an exemplary embodiment. As shown in FIG. 12, looking from the side of the light-emitting apertures of the first chip 1030, the first chip 1030 comprises a plurality of blocks (A₁₋₁-A₁₀₋₈) arranged orderly. In this exemplary embodiment, the blocks (A₁₋₁-A₁₀₋₈) are arranged into a two-dimensional (2D) array, such as, but not limited to, a 4×6 array, an 8×10 array, a 12×16 array, or a 16×20 array. As shown in FIG. 12, in this exemplary embodiment, the blocks (A₁₋₁-A₁₀₋₈) are arranged into an 8×10 array.

As shown in FIG. 12, in this exemplary embodiment, each of the blocks (such as block A₁₋₁) of the first chip 1030 comprise a plurality of columnar structures P, and the number of the columnar structures P in one of the blocks is identical to the number of the columnar structures P in another one of the blocks. As a result, the lighting patterns and intensities of all blocks (A₁₋₁-A₁₀₋₈) of the first chip 1030 are substantially identical. For example, in this exemplary embodiment, each of the blocks (A₁₋₁-A₁₀₋₈) comprises 18 columnar structures P, and a distance between any two neighboring ones of the columnar structures P is identical to a distance between any other two neighboring ones of the columnar structures P.

As shown in FIG. 12, a first pitch d1 exists between two neighboring blocks A₁₋₁, A₁₋₂. The first pitch d1 is defined as the distance between corresponding columnar structures P in two neighboring ones of the blocks. As shown in FIG. 12, in this exemplary embodiment, the first pitch d1 is defined as the distance between the columnar structure P11 in the block A₁₋₁ and the columnar structure P21 in the block A₁₋₂. More specifically, in this embodiment, the first pitch d1 refers to the distance between the leftmost wall of the columnar structure P11 and the leftmost wall of the columnar structure P21.

Please also refer to FIG. 10 and FIG. 11. The first chip 1030 comprises a first conduction structure 902 and a second conduction structure 904. The columnar structures P in the blocks (A₁₋₁-A₁₀₋₈) are electrically connected to the first conduction structure 902 and the second conduction structure 904. Alternatively, in some embodiments, the first chip 1030 may comprise a plurality of pairs of first and second conduction structures, and the columnar structures in different blocks may be electrically connected to the first conduction structures and the second conduction structures of different pairs of conduction structures, so that the blocks of the first chip 1030 can be independently controlled to realize divided lighting control and thus increase the application potential of the chip package structure according to one or some embodiments. For example, by utilizing different pairs of conduction structures, the block A₁₋₁ and the block A₁₋₂ can be independently controlled so that the block A₁₋₁ and the block A₁₋₂ do not emit light simultaneously, and the columnar structures in the same block (the columnar structures in the block A₁₋₁ or the columnar structures in the block A₁₋₂), which are the light-emitting apertures of the VCSEL chip, emit light simultaneously.

The following paragraphs will illustrate the details of the second chip of the chip package structure according to one or some embodiments.

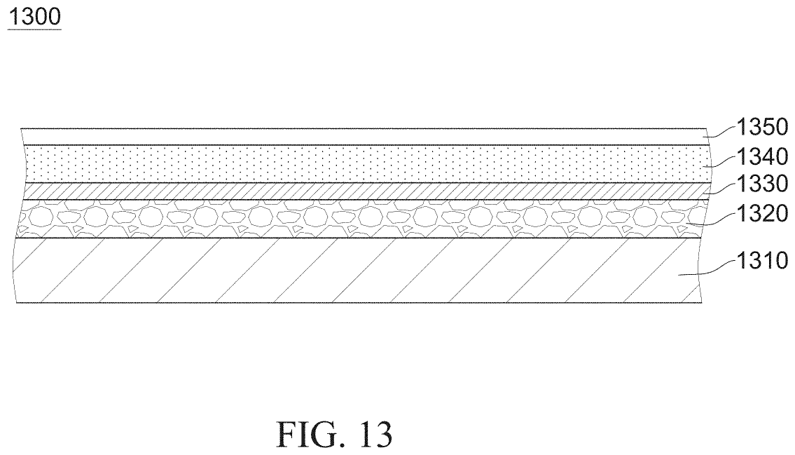

Please refer to FIG. 13. FIG. 13 illustrates a schematic cross-sectional view of the basic epitaxial structure 1300 of a second chip of a chip package structure according to an exemplary embodiment. As previously illustrated, the second chip may be a photovoltaic chip or a photodiode chip. The basic structure of the second chip comprises a substrate 1310, a first semiconductor structure 1320, a first active layer 1330, a second semiconductor structure 1340, and a transmission layer 1350, in which the first semiconductor structure 1320, the first active layer 1330, the second semiconductor structure 1340, and the transmission layer are sequentially on the substrate 1310, as shown in FIG. 13. In this exemplary embodiment, the transmission layer 1350 on the second semiconductor structure 1340 is adapted to increase current spreading capability and effectively capture the electricity generated by the active layer 1330, so that the energy conversion efficiency of the second chip is increased, but the disclosure is not limited thereto.

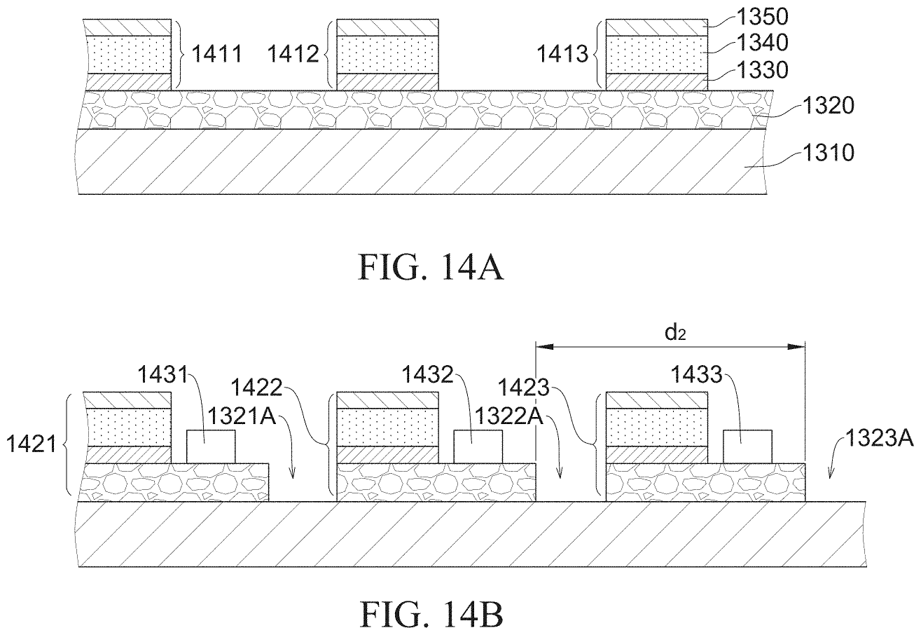

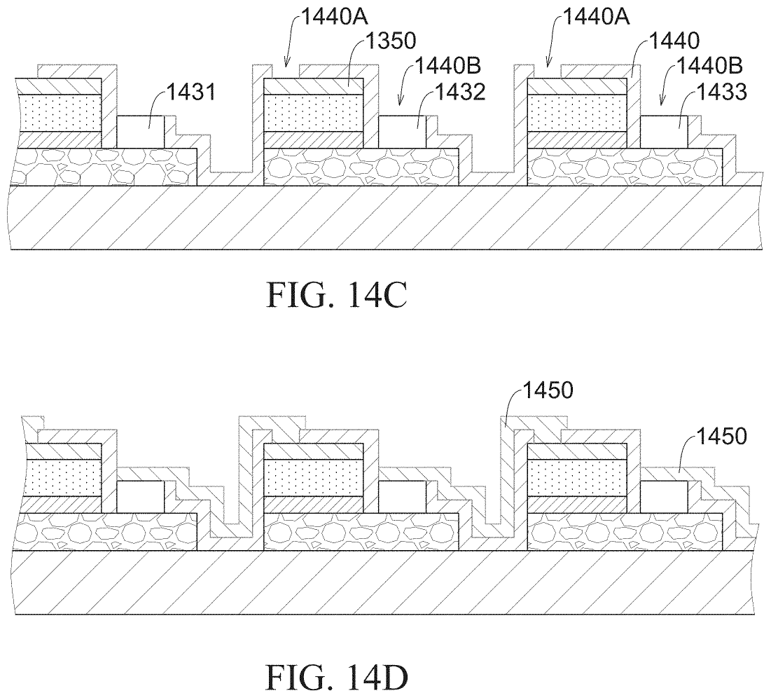

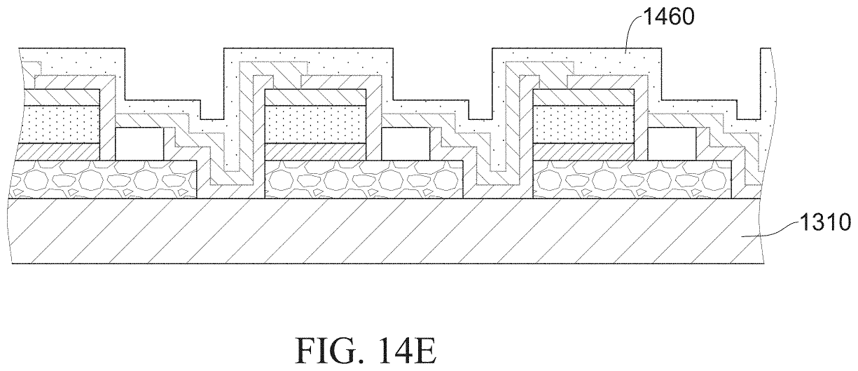

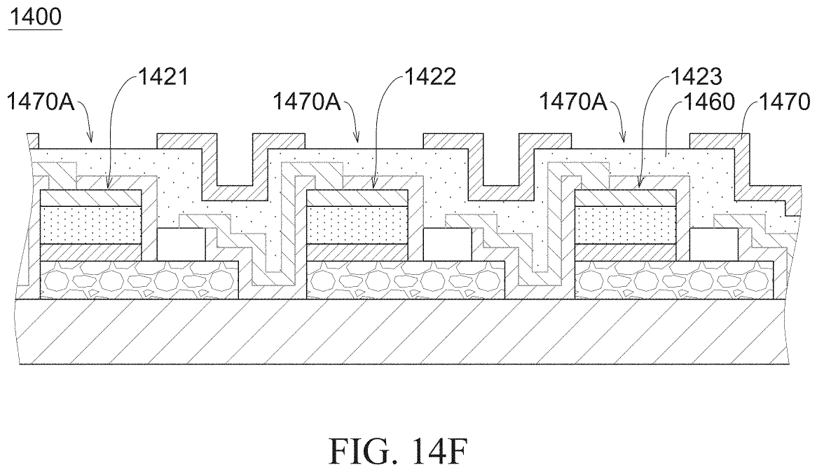

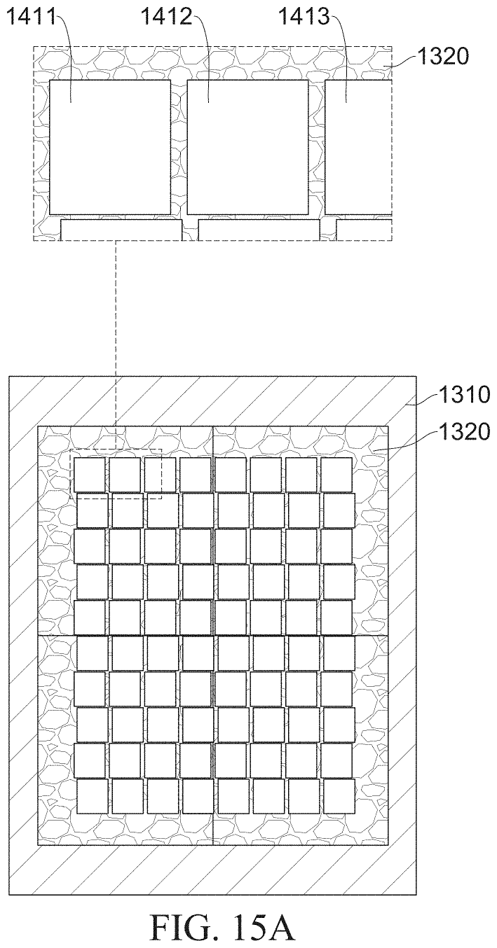

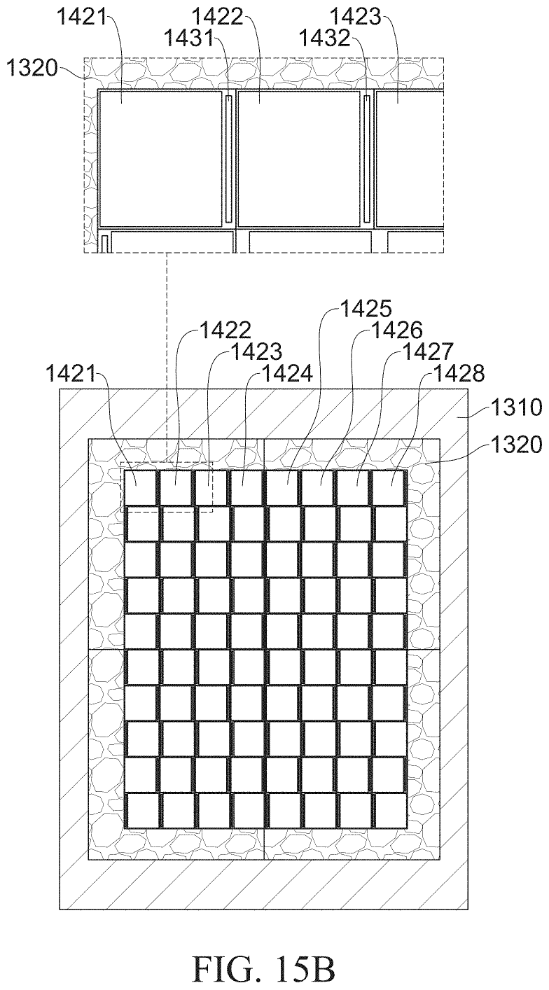

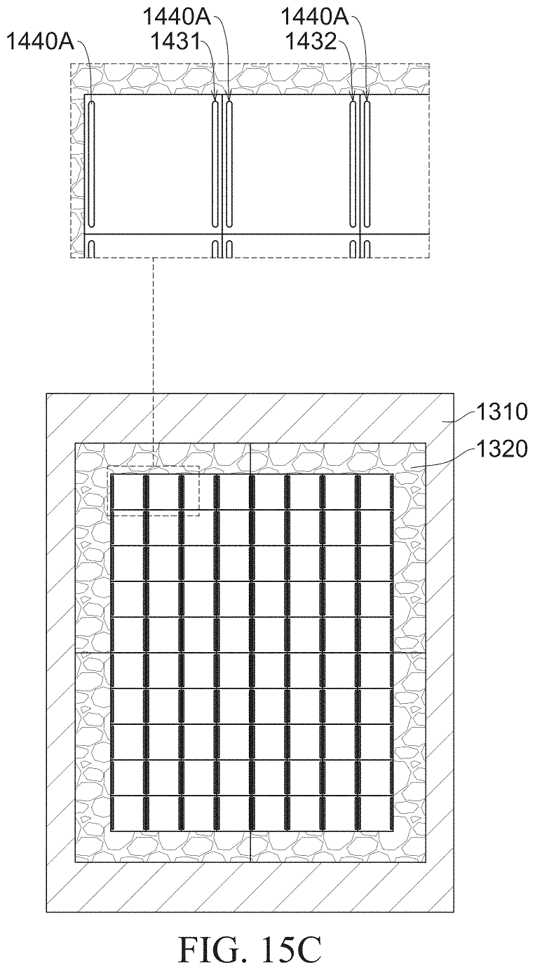

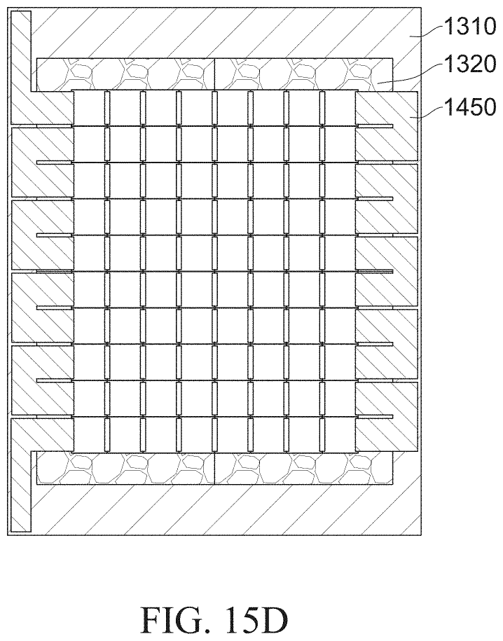

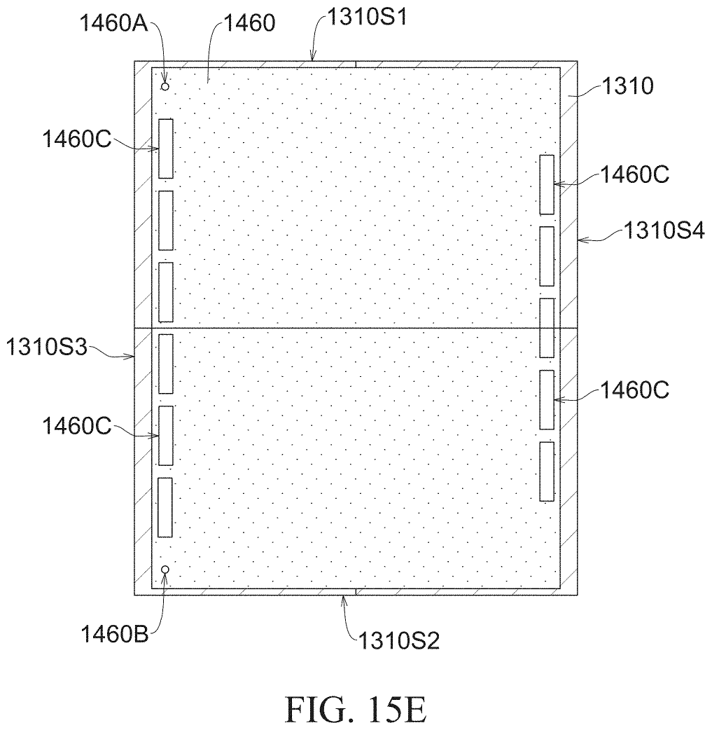

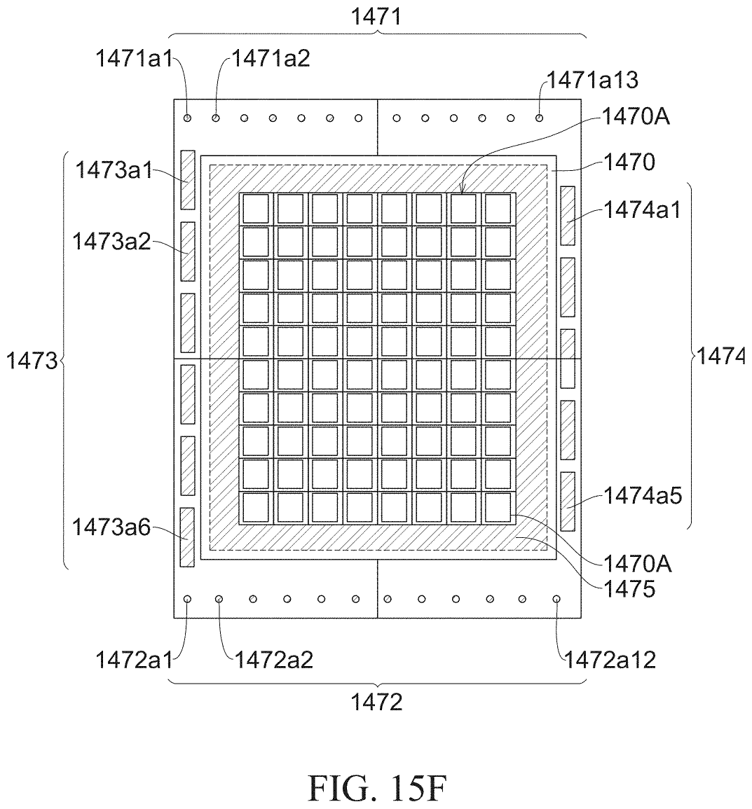

Please refer to FIG. 14A through FIG. 14F and FIG. 15A through FIG. 15F for the following illustration on the manufacture process of the second chip in the chip package structure according to one or some embodiments.

Please refer to FIG. 14A through FIG. 14A. FIG. 14A through FIG. 14F illustrate schematic cross-sectional views of the structures of the second chip shown in FIG. 13 in all manufacture steps of the manufacturing process of the second chip according to an exemplary embodiment. FIG. 15A through FIG. 15F illustrate schematic top views of the structures of the second chip in all manufacture steps of the manufacturing process of the second chip corresponding to FIG. 14A through FIG. 14F.

In this exemplary embodiment, the second chip is a photovoltaic chip and is adapted to receive the light emitted by the first chip and then convert the light into electricity output. As previously illustrated, the basic structure 1300 of the second chip shown in FIG. 13 comprises a substrate 1310, a first semiconductor structure 1320, a first active layer 1330, a second semiconductor structure 1340, and a transmission layer 1350, in which the first semiconductor structure 1320, the first active layer 1330, the second semiconductor structure 1340, and the transmission layer 1350 are sequentially on the substrate 1310.

First, as shown in FIG. 14A, the first active layer 1330, the second semiconductor structure 1340, and the transmission layer 1350 of the basic structure 1300 are partially removed, so that the first semiconductor structure 1320 is partially exposed, and a plurality of first mesa structures (such as the first mesa structures 1411-1413) is formed. Each of the first mesa structures 1411-1413 is formed by the unremoved parts of the first active layer 1330, the second semiconductor structure 1340, and the transmission layer 1350. According to the top view shown in FIG. 15A, the mesa structures 1411-1413 are arranged into a 2D array (such as, but not limited to, a 4×6 array, an 8×10 array, a 12×16 array, a 16×20 array) with an orderly misalignment. In this exemplary embodiment, the first mesa structures are arranged into an 8×10 array.

Next, as shown in FIG. 14B, the first semiconductor structure 1320 is partially removed to form a plurality of grooves 1321A-1323A, so that the portions of the substrate 1310 beneath the grooves 1321A-1323A are exposed through the grooves 1321A-1323A, and a plurality of second mesa structures is formed (such as the second mesa structures 1421-1423). A corresponding groove separates two neighboring ones of the second mesa structures. For example: neighboring second mesa structures 1421, 1422 are separated by the groove 1321A, and neighboring second mesa structures 1422, 1423 are separated by the groove 1322A. Each of the second mesa structures 1421-1423 comprises a first semiconductor structure 1320 and a corresponding one of the first mesa structures 1411-1413 on the first semiconductor structure 1320. In each of the second mesa structures 1421-1423, a corresponding one of first connection structures 1431-1433 is formed on the first semiconductor structure 1320. The distance between two neighboring ones of the second mesa structures (such as the second mesa structure 1422 and the second mesa structure 1423) is defined as a second pitch d2. Specifically, in this embodiment, the second pitch d2 refers to the distance between the rightmost wall of any of the second mesa structures (for example, the second mesa structure 1422) and the rightmost wall of a neighboring one of the second mesa structures (for example, the second mesa structure 1423). According to the top view shown in FIG. 15B, the second mesa structures 1421-1423 are arranged into a 2D array (such as, but not limited to, a 4×6 array, an 8×10 array, a 12×16 array, a 16×20 array) with an orderly misalignment. In this exemplary embodiment, the second mesa structures are arranged into an 8×10 array.

As previously illustrated, in one or some embodiments of the disclosure, the first chip and the second chip are connected to form the chip package structure, wherein each of the second mesa structures of the second chip is aligned with a corresponding one of the blocks (for example, the blocks A₁₋₁-A₁₀₋₈ of the first chip 1030 shown in FIG. 12) of the first chip. Specifically, in this exemplary embodiment, each of the second mesa structures 1421-1423 is aligned with each of the blocks A₁₋₁-A₁₋₃ of the first chip 1030 shown in FIG. 12, and the first pitch d1 of the first chip 1030 and the second pitch d2 of the second chip are substantially identical. Alternatively, in some other exemplary embodiments, the second pitch d2 may be greater or less than the first pitch d1.

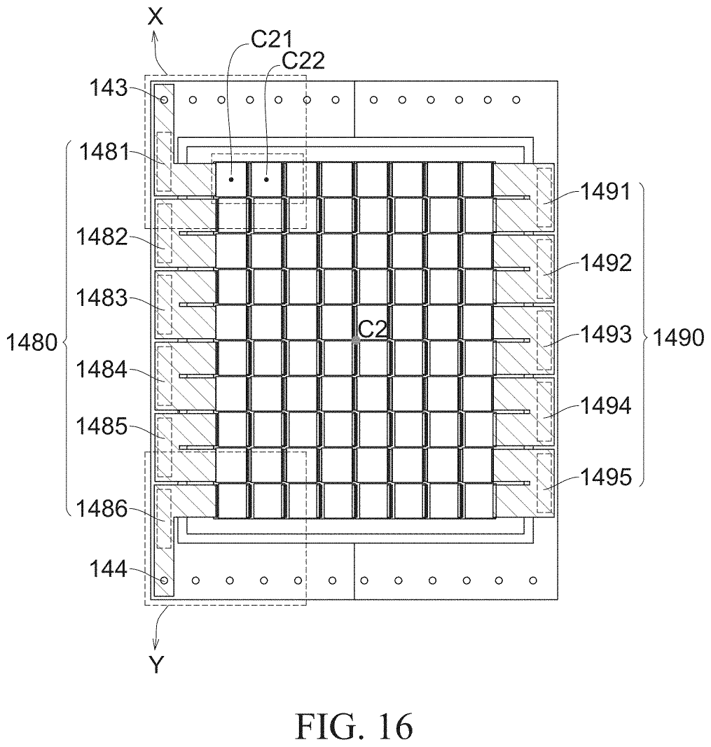

As shown in FIG. 12, in an exemplary embodiment, the first chip 1030 has a geometric center C1, and each of the blocks A₁₋₁-A₁₀₋₈ has a corresponding geometric center as well, such as geometric centers C11, C12. After the first chip and the second chip are connected and packaged, the geometric center C1 of the first chip and the geometric center of the second chip (C2 shown in FIG. 16) are substantially aligned with each other, with an error range of ±20 μm. In another exemplary embodiment, preferably, each of the geometric centers C11, C12, of the blocks of the first chip 1030 is substantially aligned with a corresponding one of the geometric centers (such as the geometric centers C21, C22 shown in FIG. 16) of the second mesa structures of the second chip. Regarding this alignment configuration, the following paragraphs will further illustrate using FIG. 16. In an exemplary embodiment, according to the top view, the horizontal distance between the geometric center C1 of the first chip and the geometric center of the second chip is less than 30 μm, such as in a range between 5 μm and 30 μm, and the horizontal distance between the geometric center C11 of the first block and the geometric center (such as C21 shown in FIG. 16) of the corresponding one of the second mesa structures is also less than 30 μm, such as in a range between 5 μm and 30 μm.

Next, as shown in FIG. 14C and FIG. 15C, a first insulating layer 1440 is formed. The first insulating layer 1440 covers the sides and top of each of the second mesa structures 1421-1423 and covers the first semiconductor 1320 and the substrate 1310. The first insulating layer 1440 comprises a plurality of first openings 1440A and a plurality of second openings 1440B. The portions of the transmission layer 1350 beneath the first openings 1440A are exposed through the first openings 1440A, and the portions of the first connection structures 1431-1433 beneath the second openings 1440B are exposed through the second openings 1440B.

Next, as shown in FIG. 14D and FIG. 15D, a connection layer 1450 is formed. The connection layer 1450 covers the first insulating layer 1440, fills the first openings 1440A, and covers the second openings 1440B, so that the connection layer 1450 connects the transmission layer 1350 to the first connection structures 1431-1433. The second mesa structures 1421-1423 are thus electrically connected to each other. Specifically, in this embodiment, the connection layer 1450 connects the first connection structure of one of the second mesa structures (for example, the connection structure 1432 of the second mesa structure 1422) to the first opening of a neighboring one of the mesa structures (for example, the first opening 1440A of the second mesa structure 1423), so as to form the electrical connections among the second mesa structures. In this exemplary embodiment, the second mesa structures 1421-1423 are connected in series, so as to realize the high output voltage feature of the chip package structure. In some other exemplary embodiments, depending on actual applications of the chip package structure, the second mesa structures may be connected in parallel, so as to realize the large current output feature of the chip package structure.

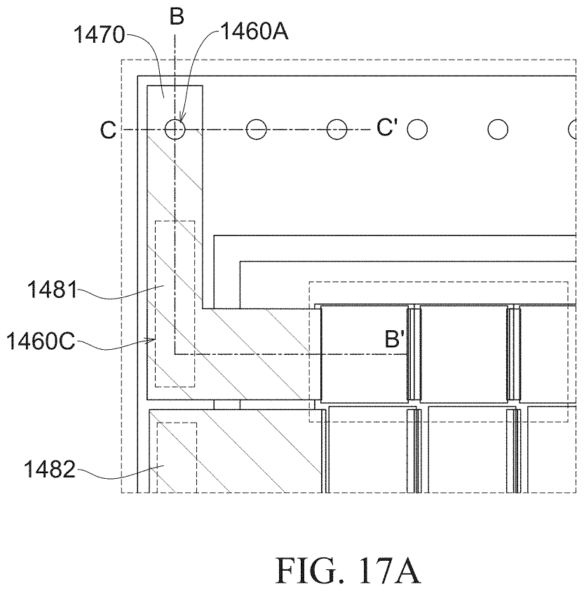

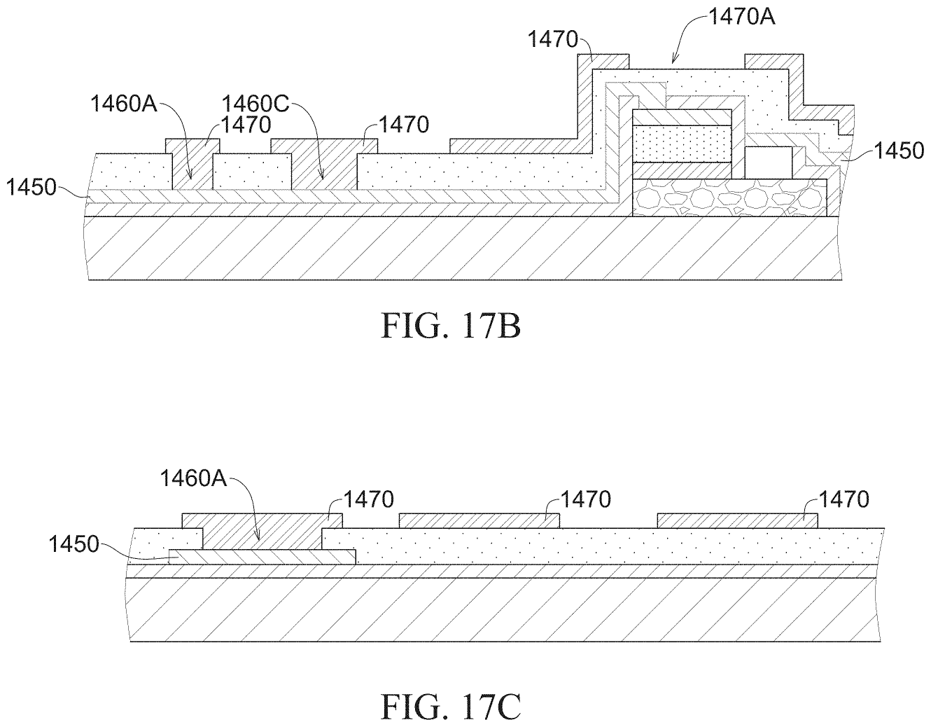

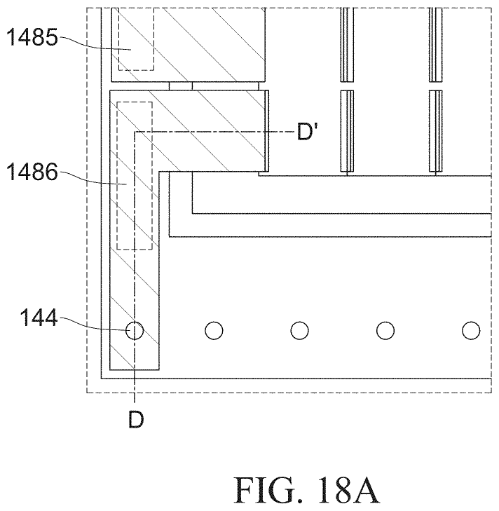

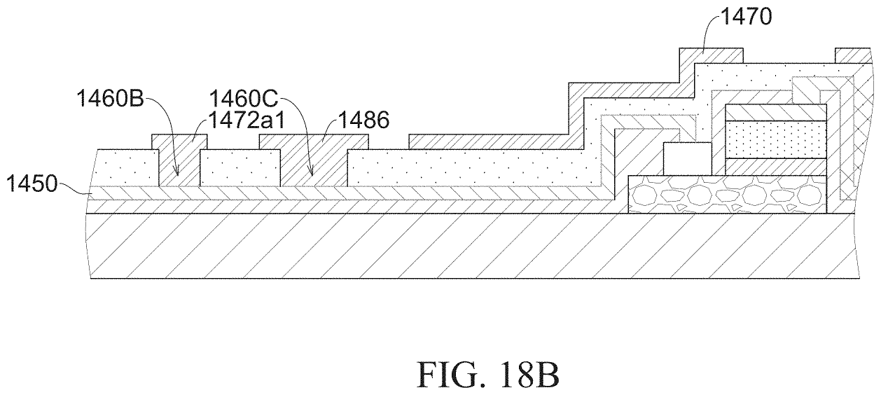

Next, as shown in FIG. 14E, a second insulating layer 1460 is formed. The second insulating layer 1460 covers the connection layer 1450. According to the top view shown in FIG. 15E, the second insulating layer 1460 comprises a third opening 1460A, a fourth opening 1460B, and a plurality of fifth openings 1460C, so that portions of the connection layer 1450 beneath the third opening 1460A, portions beneath the fourth opening 1460B, and portions beneath the fifth openings 1460C are exposed (will be further illustrated with FIG. 17A through FIG. 17C and FIG. 18A through FIG. 18C) through the third opening 1460A, the fourth opening 1460B, and the fifth openings 1460C, respectively. Except the regions of the second mesa structures corresponding to the third opening 1460A, the fourth opening 1460B, and the fifth openings 1460C, the second insulating layer 1460 covers most of the surface regions of the second mesa structures 1421-1423.

In this exemplary embodiment, according to the top view, the substrate 1310 is rectangular-shaped. The substrate 1310 has a first side 1310S1 and a second side 1310S2 opposite to each other as well as a third side 1310S3 and a fourth side 1310S4 opposite to each other. The length of the first side 1310S1 and the length of the second side 1310S2 are less than the length of the third side 1310S3 and the length of the fourth side 1310S4. In other words, in this embodiment, the first side 1310S1 and the second side 1310S2 are the shorter sides of the rectangular substrate 1310, while the third side 1310S3 and the fourth side 1310S4 are the longer sides of the rectangular substrate 1310. As shown in FIG. 15E, in this exemplary embodiment, the third opening 1460A of the second insulating layer 1460 is adjacent to a joint portion between the first side 1310S1 and the third side 1310S3, and the fourth opening 1460B is adjacent to a joint portion between the second side 1310S2 and the third side 1310S3; while the fifth openings 1460C are divided into two groups, one group of the fifth openings 1460C are arranged adjacent to and along the third side 1310S3, and the other group of the fifth openings 1460C are arranged adjacent to and along the fourth side 1310S4. A first output electrode set 1480 and a second output electrode set 1490 are thus further formed (will be illustrated later).

Next, as shown in FIG. 14, an electrode layer 1470 is formed. The electrode layer 1470 covers the second insulating layer 1460 to achieve the formation of the second chip 1400 in this exemplary embodiment. According to the top view shown in FIG. 15F, the electrode layer 1470 is formed to comprise a first electrode portion 1471, a second electrode portion 1472, a third electrode portion 1473, and a fourth electrode portion 1474, which are respectively arranged adjacent to and along the first side 1310S1, the second side 1310S2, the third side 1310S3, and the fourth side 1310S4 of the rectangular substrate 1310. As shown in FIG. 15F, the first electrode portion 1471 comprises a plurality of first electrode connection structures 1471a1-1471a13 (in this embodiment, the number of the first electrode connection structures 1471a1-1471a13 is thirteen), the second electrode portion 1472 comprises a plurality of second electrode connection structures 1472a1-14722a12 (in this embodiment, the number of the second electrode connection structures 1472a1-14722a12 is twelve), the third electrode portion 1473 comprises a plurality of third electrode connection structures 1473a1-1473a6 (in this embodiment, the number of the third electrode connection structures 1473a1-1473a6 is six), and the fourth electrode portion 1474 comprises a plurality of fourth electrode connection structures 1474a1-1474a5 (in this embodiment, the number of the fourth electrode connection structures 1474a1-1474a5 is five).

The electrode layer 1470 further comprises a fifth electrode portion in a central region enclosed by the first electrode portion 1471, the second electrode portion 1472, the third electrode portion 1473, and the fourth electrode portion 1474. The fifth electrode portion 1475 covers the second mesa structures (such as the second mesa structures 1421-1423) and the grooves (such as the groove 1321A-1323A) The fifth electrode portion 1475 comprises a plurality of sixth openings 1470A. The portions of the second insulating layer 1460 beneath the sixth openings 1470A are exposed through the sixth openings 1470A. The sixth openings 1470A are arranged orderly into a 2D array which corresponds to the second mesa structures. In this exemplary embodiment, a light passes through the sixth openings 1470A, enters the insulating layer 1460 and the second mesa structures 1421-1423, and is then converted into electricity; that is, the sixth openings 1470A define a plurality of light receiving regions of the second chip 1400.

Please refer to FIG. 16. FIG. 16 illustrates a schematic top view of the second chip according to an exemplary embodiment. Specifically, FIG. 16 illustrates the top view of the second chip 1400 formed by the aforementioned manufacturing process. For illustrative purposes, some structures and/or layers (such as the first insulating layer, the second insulating layer, and the second mesa structures) of the second chip 1400 are not shown in FIG. 16 to make the view clearer.

Please also refer to FIG. 17A through FIG. 17C, FIG. 18A, and FIG. 18B. FIG. 17A illustrates an enlarged partial view of FIG. 16 and is used to illustrate the structure within the block X enclosed by dotted line, and FIG. 17B and FIG. 17C illustrate schematic cross- sectional views of FIG. 17A along line B-B′ and line C-C′, respectively. FIG. 18A illustrates an enlarged partial view of FIG. 16 and is used to illustrate the structure within the block Y enclosed by dotted line, and FIG. 18B illustrates a schematic cross-sectional view of FIG. 18A along line D-D′.

In this exemplary embodiment, the first electrode connection structure 1471a1 of the electrode layer 1470 is filled in the third opening 1460A and thus the first electrode connection structure 1471a1 is connected to the connection layer 1450 to form electrical connection, so that the first conductive connection structure (such as the aforementioned first conductive connection structure 143) of the chip package structure according to one or some embodiments of the disclosure is thus formed, as shown in FIG. 17A and FIG. 17B. The second electrode connection structure 1472a1 of the electrode layer 1470 is filled in the fourth opening 1460B and thus the second electrode connection structure 1472a1 is connected to the connection layer 1450 to form electrical connection, so that the second conductive connection structure (such as the aforementioned second conductive connection structure 144) of the chip package structure according to one or some embodiments of the disclosure is thus formed, as shown in FIG. 18A and FIG. 18B. The third electrode connection structures 1473a1-1473a6 and the fourth electrode connection structures 1474a1-1474a5 of the electrode layer 1470 are filled in the fifth openings 1460C adjacent to the third side 1310S3 and adjacent to the fourth side 1310S4, respectively, and thus the third electrode connection structures 1473a1-1473a6 and the fourth electrode connection structures 1474a1-1474a5 are connected to the connection layer 1450 to form electrical connection, so that the first output electrode set 1480 and the second output electrode set 1490 are thus formed to allow the second chip 1400 to adjust output voltage based on different application scenarios. The first output electrode set 1480 is adjacent to the third side 1310S3 and comprises a plurality of first output electrodes 1481-1486 (in this embodiment, the number of the first output electrodes 1481-1486 is six), and the second output electrode set 1490 is adjacent to the fourth side 1310S4 and comprises a plurality of second output electrodes 1491-1495 (in this embodiment, the number of the second output electrodes 1491-1495 is five). For example, each of the second mesa structures (such as the second mesa structures 1421-1423 shown in FIG. 15B) is adapted to generate a voltage of 1V, and the second mesa structures are connected in series. When the second chip 1400 is connected externally to the application environment through the first output electrode 1481 and the second output electrode 1491, the output voltage of the second chip 1400 is 8 V, because eight of the second mesa structures 1421-1428 shown in FIG. 15B are connected in series. When the second chip 1400 is connected externally to the application environment through the first output electrode 1481 and the second output electrode 1492, the output voltage of the second chip 1400 is 24 V, because twenty-four of the second mesa structures 1421-1428 shown in FIG. 15B are connected in series. Alternatively, in some other exemplary embodiments, the third electrode portion 1473 (the third electrode connection structures 1473a1-1473a6) and the fourth electrode portion 1474 (the fourth electrode connection structures 1474a1-1474a5) may be omitted. The fifth electrode portion 1475 of the electrode layer 1470 is electrically insulated from the first electrode portion 1471, the second electrode portion 1472, the third electrode portion 1473, and the fourth electrode portion 1474. The fifth electrode portion 1475 of the electrode layer 1470 is beneficial for the heat dissipation of the second chip 1400 and can avoid crosstalk effect among neighboring ones of the second mesa structures.

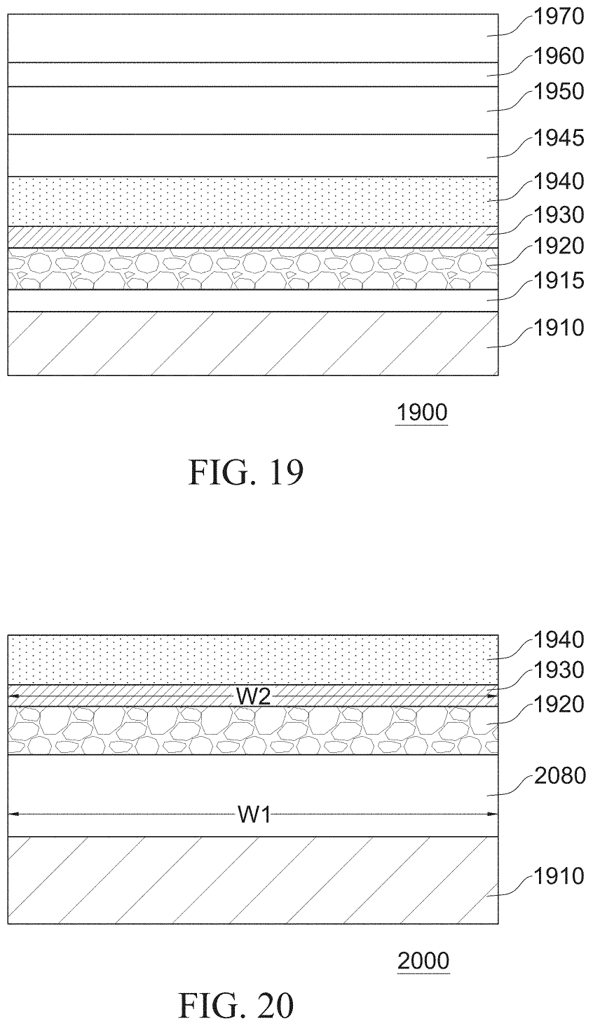

Please refer to FIG. 19. FIG. 19 illustrates a schematic view of the epitaxial structure of a second chip according to an exemplary embodiment. As shown in FIG. 19, beside the first semiconductor structure 1920, the first active structure 1930 and the second semiconductor 1940 which are sequentially on the substrate 1910, in this exemplary embodiment, the second chip 1900 further comprises a third semiconductor structure 1950, a second active structure 1960, and a fourth semiconductor structure 1970 sequentially stacked on the second semiconductor structure 1940. In this exemplary embodiment, the second chip further comprises a tunneling layer 1945 between the second semiconductor structure 1940 and the third semiconductor structure 1950. Compared with the previous exemplary embodiments, such as the second chip 1400 shown in FIG. 15F, in this exemplary embodiment, the second chip 1900 has two active structures (the first active structure 1930 and the second active structure 1940) and thus can achieve even higher energy conversion efficiency and even higher output voltage. In some other exemplary embodiments, the second chip may also comprise three or more active structures so as to absorb lights with the same or different wavelengths. However, the thickness of each of the active layers grows greater from the receiving surface of the first chip toward the substrate surface of the second chip, so that the current generated by each of the active layers through light absorption is identical to one another. In some other exemplary embodiments, the thickness of the first active layer 1930 may be in a range between 3 μm and 10 μm; this range of thickness is beneficial for the absorption of the light emitted by the first chip (not shown in the figure). The second chip 1900 may further comprise a current cutoff layer 1915 between the substrate 1910 and the first semiconductor structure 1920. The current cutoff layer is made of a non-doped semiconductor material such as InGaP, so that current leakage is avoided. The current cutoff layer 1915 may also be used as an etch stop layer for the removal of the substrate 1910, and other substrates can be bonded to the other side of the substrate 1910 through wafer bonding. The other substrates may be made of electrically conductive or electrically insulating material(s). Wafer bonding materials will exist between the substrate and the semiconductor layer to provide electrical insulation.

Please refer to FIG. 20. FIG. 20 illustrates a schematic view of the epitaxial structure of a second chip according to an exemplary embodiment. As shown in FIG. 20, the second chip 2000 may comprise a reflective structure 2080 between the substrate 1910 and the first semiconductor structure 1920. The reflective structure 2080 is beneficial for the realization of the second chip with even higher energy conversion efficiency. In this exemplary embodiment, the width W1 of the first active layer 1930 is roughly identical to the width W2 of the reflective structure 2080.

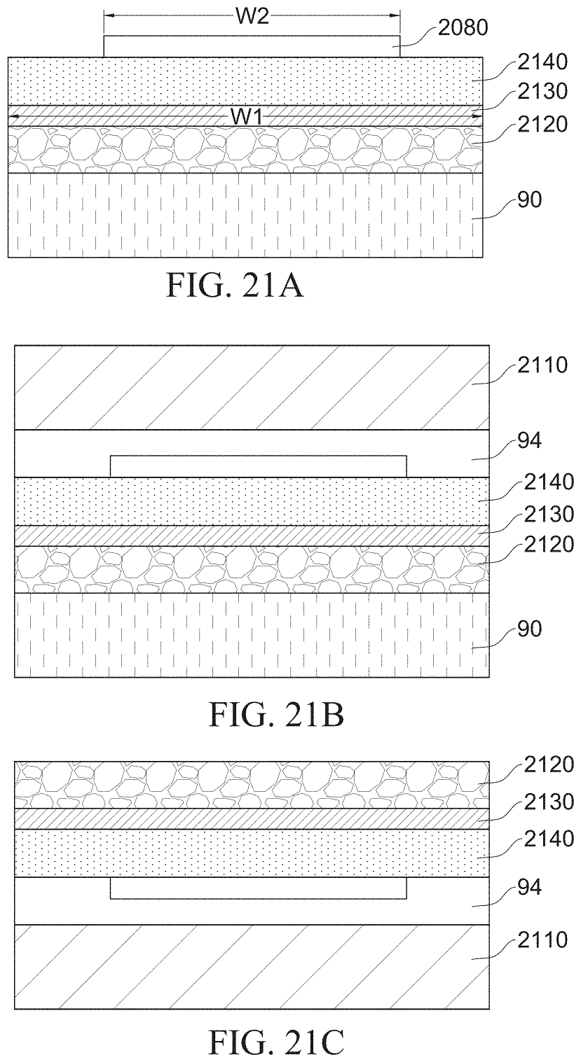

Please refer to FIG. 21Athrough FIG. 21C. FIG. 21Athrough FIG. 21C illustrate the manufacture process of a second chip according to an exemplary embodiment. FIG. 21A through FIG. 21C are used to illustrate the manufacture process of the second chip.

As shown in FIG. 21A, a first semiconductor structure 2120, a first active structure 2130, and a second semiconductor structure 2140 are sequentially formed on a growth substrate 90, and the reflective structure 2080 is deposited on the second semiconductor structure 2140. The width W2 of the reflective structure 2080 is less than the width W1 of the first active structure 2130.

As shown in FIG. 21B, a bonding layer 94 is formed. The bonding layer 94 covers the reflective structure 2080 and partially covers the second semiconductor 2140, so that the second semiconductor structure 2140 is connected to the substrate 2110 (i.e., the permanent substrate).

As shown in FIG. 21C, the growth substrate 90 is removed. In this exemplary embodiment, the reflective structure 2080 may be made of a metal or an alloy with high reflectivity. In an exemplary embodiment, the reflective structure 2080 comprises a light-transmissive conduction layer and/or a distributed Bragg reflective (DBR) structure.

According to one or some embodiments of the disclosure, the first and second semiconductor structures of the aforementioned second chip are of different types. For example, the first semiconductor structure is N-type, and the second semiconductor structure is P-type, or vice versa. As known by persons skilled in the art, a P-type semiconductor structure has holes as its main carrier, and an N-type semiconductor structure has electrons as its main carrier.

The active structure is the light absorbing region of the second chip. The wavelength of the absorbed light is determined by the material (or bandgap) of the active structure. In other words, the active structure is adapted to absorb light with optical energy greater than the bandgap of the active structure. For example, the bandgap of the active structure may be 0.72 eV-1.77 eV (corresponding to infrared lights with wavelengths between 700 nm and 1700 nm), 1.77 eV-2.03 eV (corresponding to red lights with wavelengths between 610 nm and 700 nm), 2.1 eV-2.175 eV (corresponding to yellow lights with wavelengths between 570 nm and 590 nm), 2.137 eV-2.48 eV (corresponding to green lights with wavelengths between 500 nm and 580 nm), 2.53 eV-3.1 eV (corresponding to blue violet or blue lights with wavelengths between 400 nm and 490 nm), or 3.1 eV-4.96 eV (corresponding to ultraviolet lights with wavelengths between 250 nm and 400 nm). In one or some embodiments of the disclosure, the active structure is a semiconductor layer comprising a dopant, wherein the concentration of the dopant in the active structure is less than the concentration of the dopant in the first semiconductor structure and/or the concentration of the dopant in the second semiconductor structure. Specifically, in this embodiment, the concentration of the dopant in the active layer is less than 5×10¹⁶ cm⁻³, such as between 1×10¹⁵ cm⁻³ and 5×10¹⁶ cm⁻³. In an exemplary embodiment, the first active structure and the first semiconductor structure are of the same type, or the first active structure and the first semiconductor structure have the same dopant. In another exemplary embodiment, the first active structure is a semiconductor without intentional doping.

In an exemplary embodiment, the active structure of the second chip is adapted to absorb red lights with wavelengths between 750 nm and 1100 nm. In an exemplary embodiment, the first active structure is a single layer between the first semiconductor structure and the second semiconductor structure. In another exemplary embodiment, the first semiconductor structure directly contacts the second semiconductor structure, wherein the first active structure is the interface between the first semiconductor structure and the second semiconductor structure.

The permanent substrate (such as the substrate 1910) of the second chip is adapted to support the first semiconductor structure and the other layers formed on the first semiconductor structure. The first semiconductor structure, the active structure, and the second semiconductor structure may be formed on the substrate or the growth substrate using epitaxial growth techniques such as metal-organic chemical vapor deposition (MOCVD), molecular beam epitaxy (MBE), or hydride vapor phase epitaxy (HYPE). When forming the layered structures on the growth substrate, substrate transferring technology can be used, so that the layered structures can be transferred to the permanent substrate using the bonding layer (such as the bonding layer 94 shown in FIG. 21B), and the growth substrate can be optionally removed. The permanent substrate may be with dopants or without dopants, may be N-type or P-type, and may be made of glass, sapphire, silicon carbide, silicon, ceramics, or metal(s).

In an exemplary embodiment, the transmission layer has a higher transmittance for the light which enters the second chip and has a wavelength that falls within the target range of wavelength. For example, the transmission layer may be semiconductor materials, metals, metal alloys, metal oxides, diamond-like carbon (DLC), or graphene. For example, the metal oxide may be ITO, InO, SnO, CTO, ATO, AZO, ZTO, GZO, IWO, ZnO, or IZO. For example, the metal may be Cu, Al, Cr, Sn, Au, Ni, Ti, Pt, Pd, Zn, Cd, Sb, Co, Ge, Be, or an alloy thereof.

In an exemplary embodiment, the materials of the first semiconductor structure 202, the second semiconductor 206, and the active structure 204 include group III-V material compound semiconductors, such as AlGaInAs series, AlGaInP series, AlInGaN series, AlAsSb series, InGaAsP series, InGaAsN series, AlGaAsP series, or the like, and the materials of the first semiconductor structure 202, the second semiconductor 206, and the active structure 204 may be, such as AlGaInP, GaAs, InGaAs, AlGaAs, GaAsP, GaP, InGaP, AlInP, GaN, InGaN, or AlGaN. In the exemplary embodiments of the disclosure, if not specifically illustrated, the abovementioned chemical formulae refer to “compounds conforming to stoichiometry” and “compounds not conforming to stoichiometry”, wherein “compounds conforming to stoichiometry” may refer to compounds in which a total stoichiometric quantity of group III elements is identical to a total stoichiometric quantity of group V elements; on the contrary, “compounds not conforming to stoichiometry” may refer to compounds in which the total stoichiometric quantity of group III elements is not identical to the stoichiometric quantity of group V elements. For example, the chemical formula AlGaInAs series refers to a compound having group III element Al and/or Ga and/or In, and group V element As, wherein the total stoichiometric quantity of group III elements (Al and/or Ga and/or In) is identical to the total stoichiometric quantity of group V elements (As). Further, if the aforementioned chemical formulae refer to compounds conforming to stoichiometry , the AlGaInAs series represents (Al_{y1}Ga_{(1-y1)})₁₋ₓ₁Inₓ₁As, wherein 0≤x1≤1, and 0≤y1≤1; the AlGaInP series represents (Al_{y2}Ga_{(1-y2)})₁₋ₓ₂Inₓ₂P, wherein 0≤x2≤1, and 0≤y2≤1; the AlInGaN series represents (Al_{y3}Ga_{(1-y3)})₁₋ₓ₃Inₓ₃N, wherein 0≤x3≤1, and 0≤y3≤1; the AlAsSb series represents AlAsₓ₄Sb₍₁₋ₓ₄₎, wherein 0≤x4≤1; the InGaAsP series represents Inₓ₅Ga₁₋ₓ₅As_{1-y4}P_{y4}, wherein 0≤x5≤1, and 0≤y4≤1; the InGaAsN series represents Inₓ₆Ga₁₋ₓ₆As_{1-y5}N_{y5}, wherein 0≤x6≤1, and 0≤y5≤1; the AlGaAsP series represents Alₓ₇Ga₁₋ₓ₇As_{1-y6}P_{y6}, wherein 0≤x7≤1, and 0≤y6≤1; and the InGaPSb series represents Inₓ₈Ga₁₋ₓ₈P_{y7}Sb_{1-y7}, wherein 0≤x8≤1, and 0≤y7≤1. In an exemplary embodiment, the materials of the first semiconductor structure, the active structure, and the second semiconductor structure are In_{z}Ga_{(1-z)}P, wherein 0<z<1. In another exemplary embodiment, the material of the first semiconductor structure may be AlGaInAs: Zn series, AlGaInP: Zn series, or InGaPSb: Zn series; the material of the second semiconductor structure may be AlGaInAs: Si series, AlGaInP: Si series, or InGaPSb: Si series, and the material of the active structure may be i-AlGaInAs series, i-AlGaInP series, or i-InGaPSb series.

In one or some embodiments of the disclosure, the connection layer/connection structure and the electrode layer of the second chip comprise a metal or comprise a transparent conductive material, and the contact points comprise a metal material. For example, the metal material may include Cu, Al, Cr, Sn, Au, Ni, Ti, Pt, Pd, Zn, Cd, Sb, Co, Be, Ge, or an alloy thereof. The transparent conductive material may be ITO, InO, SnO, CTO, ATO, AZO, ZTO, GZO, IWO, ZnO, IZO, or graphene.

The insulating layer comprises oxide or nitride, such as SiO₂, Al₂O₃, or SiNₓ. In an exemplary embodiment, the insulating layer may be a multi-layered structure. For example, the insulating layer may comprise a first layer of SiO₂ and a second layer of SiNₓ neighboring the first layer.

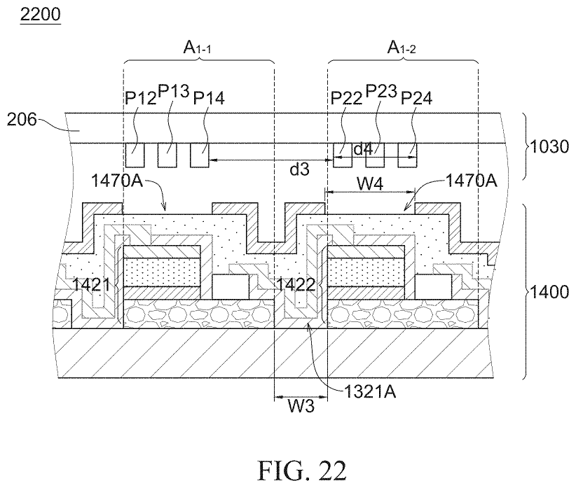

Please refer to FIG. 22. FIG. 22 illustrates a schematic cross-sectional view of a chip package structure according to an exemplary embodiment. As shown in FIG. 22, the chip package structure 2200 is inversely arranged, as compared with the chip package structure 100 shown in FIG. 1A. In this exemplary embodiment, the first chip may be a VCSEL chip, and the second chip may be a photovoltaic chip. In this exemplary embodiment, the first chip may be the first chip 1030 shown in FIG. 10, and the second chip may be the second chip 1400 shown in FIG. 14F. For clear illustration, FIG. 22 does not illustrate most structures or layers of the first chip 1030 and just illustrates the second semiconductor structure 206 and the columnar structures P (the columnar structures P12, P13, P14 in block A₁₋₁ and the columnar structures P22, P23, P24 in the block A₁₋₂ shown in FIG. 22) of the first chip 1030.

As shown in FIG. 22, the columnar structures P12, P13, P14, P22, P23, P24 of the first chip 1030 are arranged above the second mesa structures 1421, 1422 of the second chip 1400. Specifically, in this embodiment, the columnar structures P12, P13, P14 of the first chip 1030 are aligned with the second mesa structure 1421 of the second chip 1400, while the columnar structures P22, P23, P24 of the first chip 1030 are aligned with the second mesa structure 1422 of the second chip 1400. In other words, in this exemplary embodiment, the first chip 1030 and the second chip 1400 may be operated while being bonded together, and the light emitting regions (where the columnar structures P12, P13, P14 are and where the columnar structures P22, P23, P24 are) of the first chip 1030 and the light receiving regions (the second mesa structure 1421 and the second mesa structure 1422) are in a one-on-one relationship. In some embodiments, considering the space for the electrodes and wiring of the second chip 1400, the area of the light receiving regions may be slightly less than the area of the light emitting regions in the chip package structure.

In this exemplary embodiment, as shown in FIG. 22, the distance d3 between the columnar structure P14 in block A₁₋₁ and the columnar P22 in the block A₁₋₂ is greater than the width W3 of the groove 1321A. The distance d4, which is the distance between the leftmost columnar structure P12 or P22 and the rightmost columnar structure P14 or P24 in the block A₁₋₁ or the block A₁₋₂, is equal to or less than the width W4 of the sixth opening 1470A of the electrode layer 1470. In other words, in this embodiment, the light emitted by all the columnar structures P12, P13, P14, P22, P23, P24 in the blocks A₁₋₁, A₁₋₂, of the first chip 1030 can be completely received by the second mesa structures 1421, 1422.

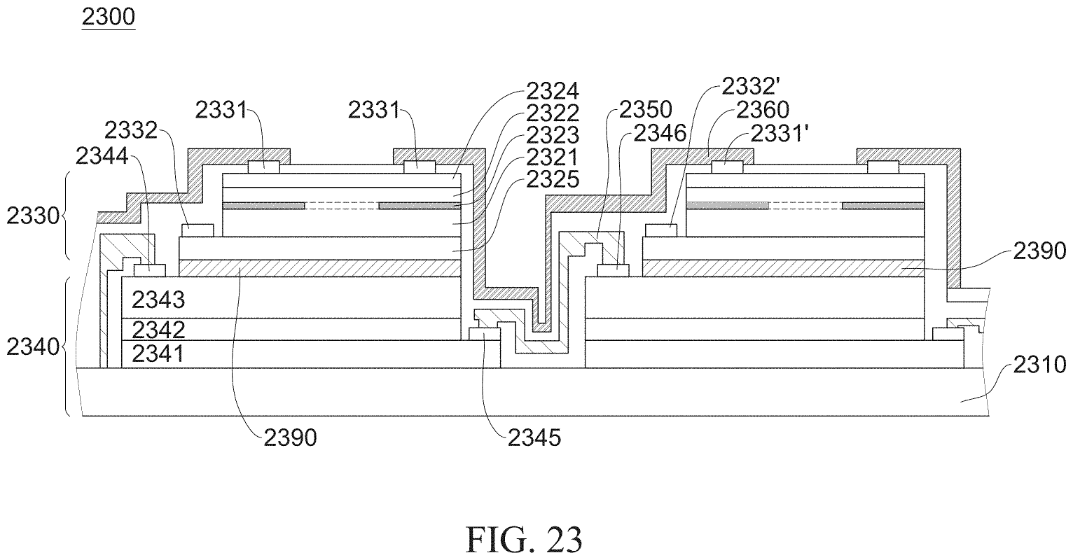

Please refer to FIG. 23. FIG. 23 illustrates a schematic cross-sectional view of a chip package structure according to yet another exemplary embodiment. As shown in FIG. 23, the chip package structure 2300 comprises a first chip 2330 and a second chip 2340 which are on the substrate 2310. The first chip 2330 and the second chip 2340 are both horizontal chips. The first chip 2330 and the second chip 2340 are connected to each other and electrically insulated from each other through the spacing layer 2390. The spacing layer 2390 and the first chip 2330 can be sequentially formed on the second chip 2340 through epitaxial growth. As previously illustrated, the material of the spacing layer 2390 is a semiconductor material with low doping concentration and high electrical resistance.

In this exemplary embodiment, the first chip 2330 may be a laser chip. As shown in FIG. 23, the first chip 2330 comprises a first semiconductor structure 2321, a second semiconductor structure 2322, and an active structure 2323 between the first semiconductor structure 2321 and the second semiconductor structure 2322. The first chip 2330 comprises a first contact layer 2324 which covers the second semiconductor structure 2322. The first contact layer 2324 is connected to the first semiconductor structure 2331 to form electrical connection. The first chip 2330 further comprises a second contact layer 2325 which connects the first semiconductor structure 2321 to the second semiconductor structure 2322 to form electrical connection.

The second chip 2340 may be a photovoltaic chip. As shown in FIG. 23, the mesa structure of the second chip 2340 comprises a first semiconductor structure 2341, an active layer 2342, a second semiconductor structure 2343, and contact points 2344, 2345, in which the contact points 2344, 2345 are adapted to form electrical connection. Due to the connection layer 2350 connecting the contact points 2345, 2346 of two neighboring mesa structures, two neighboring mesa structures of the second chip 2340 are connected in series. Moreover, due to the first connection structure 2360 connecting the first conduction structure 2331 of one of the first chips 2330 to the first conduction structure 2331′ of a neighboring one of the first chips 2330, two neighboring first chips 2330 are connected in series. In other words, in this exemplary embodiment, the first conduction structure 2331 of one of the first chips 2330 and the first conduction structure 2331′ of a neighboring one of the first chips 2330 are electrically connected through the first connection structure 2360. Similarly, the second conduction structure 2332 of one of the first chips 2330 and the first conduction structure 2332′ of a neighboring one of the first chips 2330 are electrically connected through a second connection structure (not shown in FIG. 23).

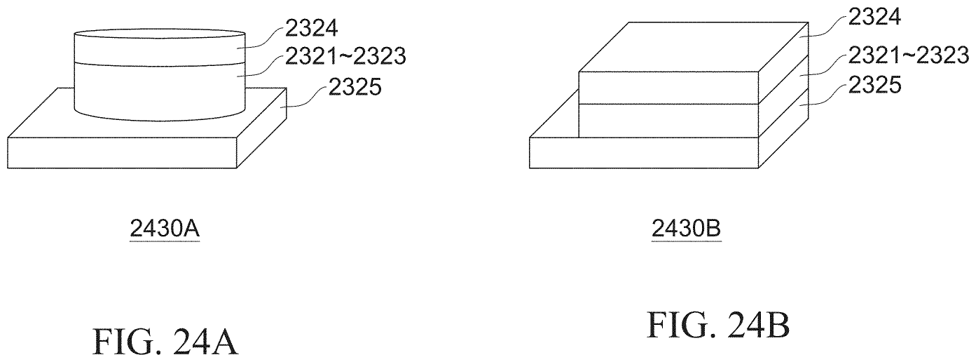

Please refer to FIG. 24A and FIG. 24B. FIG. 24A and FIG. 24B illustrate schematic perspective views showing the shapes of the chip sets of chip package structures according to two exemplary embodiments of the chip package structure, respectively. As shown in FIG. 24A and FIG. 24B, the first chip of the chip package structure in the disclosure may be columnar, such as the first chip 2430A shown in FIG. 24A, which may be a VCSEL chip, or a cube, such as the first chip 2430B shown in FIG. 24B, which may be an LED chip. As shown in FIG. 24A and FIG. 24B, the first chip 2430A shown in FIG. 24A and the first chip 2430B shown in FIG. 24B may comprise identical structures, but an effective light emitting area of the first chip 2430B shown in FIG. 24A is greater than an effective light emitting area of the first chip 2430B shown in FIG. 24B, so that higher energy conversion efficiency of the chip package structure according to one or some embodiments of the disclosure can be achieved. The material of the light emitting element according to one or some embodiments of the disclosure may be GaAs or GaN.

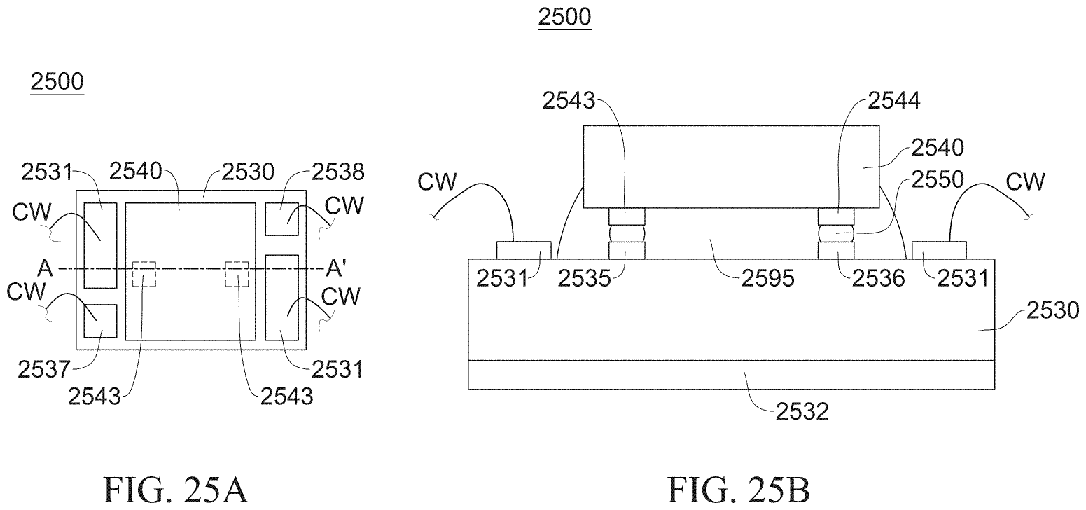

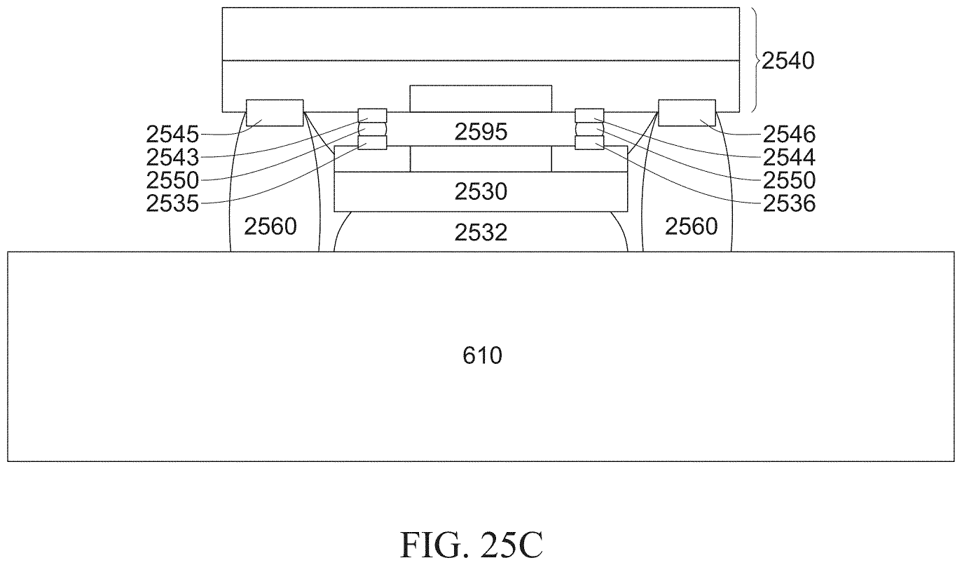

Please refer to FIG. 25A and FIG. 25B. FIG. 25A and FIG. 25B illustrate a schematic top view and a schematic cross-sectional view of a chip package structure according to an exemplary embodiment, respectively, wherein FIG. 25B is a cross-sectional view of FIG. 25A along line A-A′. In this exemplary embodiment, as shown in FIG. 25A and FIG. 25B, the first chip is a vertical chip, the second chip is a horizontal chip, and the size of the second chip is less than the size of the first chip. The chip package structure 2500 in this exemplary embodiment is configured similarly to the chip package structure 600 shown in FIG. 6, and the difference between the embodiment shown in FIG. 6 and the embodiment shown in FIG. 25A and FIG. 25B is at least that, in this exemplary embodiment, the first conduction structure 2543 and the second conduction structure 2544 of the chip package structure 2500 are on the side of the second chip 2540 which faces the first chip 2530. Besides, conductions structures 2535, 2536 are on the first chip 2530 and are respectively connected to the conduction structures 2543, 2544 of the second chip 2540 through bonding structures 2550. The conduction structures 2535, 2536 are electrically insulated from the first conduction structure 2531 and the second conduction structure 2532 of the first chip 2530 and are adapted to provide alignment for the first chip 2530 and the second chip 2540 in the chip package structure. In this exemplary embodiment, the chip package structure 2500 further comprises bonding pads 2537, 2538, and the bonding pads 2537, 2538 are electrically connected to the conduction structures 2543, 2544 and the second chip 2540. The bonding pads 2537, 2538 and the first conduction structure 2531 are respectively electrically connected to the electrical conduction posts (not shown in the figures) through corresponding wires CW. In this exemplary embodiment, the chip package structure 2500 further comprises an underfill 2595 between the first chip 2530 and the second chip 2540 to further position the first chip 2530 and the second chip 2540. In this exemplary embodiment, the underfill may be a dielectric material and/or a light-transmissive material, such as a BCB gel.

In this exemplary embodiment, the first chip 2530 and the second chip 2540 are bonded through the chip-to-wafer bonding process. During the packaging process, the bonding structure bonds a plurality of second chips to a first wafer having a plurality of first chips (not shown in the figures). Next, the underfill 2595 is applied between the first chip 2530 and the second chip 2540. Then, the chips are cut, and the obtained chip set (including the first chip 2530 and the second chip 2540) are disposed on the substrate (such as the substrate 110 shown in FIG. 1A or the substrate 610 shown in FIG. 6) to obtain the chip package structure 2500 according to one or some embodiments of the disclosure.

Please refer to FIG. 25C. FIG. 25C illustrates a schematic cross-sectional view of a chip package structure according to another exemplary embodiment. In another exemplary embodiment, the size of the first chip is less than the size of the second chip. The chip set is disposed on the substrate 610 with a flip chip configuration. As shown in FIG. 25C, the first chip is a vertical chip, the second chip is a horizontal chip, and the size of the first chip is less than the size of the second chip. The conduction structures 2535, 2536 are on the first chip 2530 and respectively electrically connected to the conduction structures 2543, 2544 of the second chip 1540 through the bonding structures 2550. The conduction structures 2545, 2546 are on the second chip 2540 and electrically connected to the substrate 610 through the bonding structure 2560. In this exemplary embodiment, the underfill 2595 is further between the first chip 2530 and the second chip 2540 to further position the first chip 2530 and the second chip 2540. In this exemplary embodiment, the underfill may be a dielectric material and/or a light-transmissive material, such as a BCB gel.

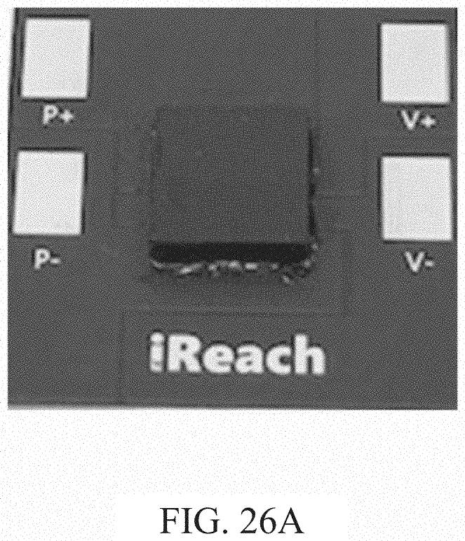

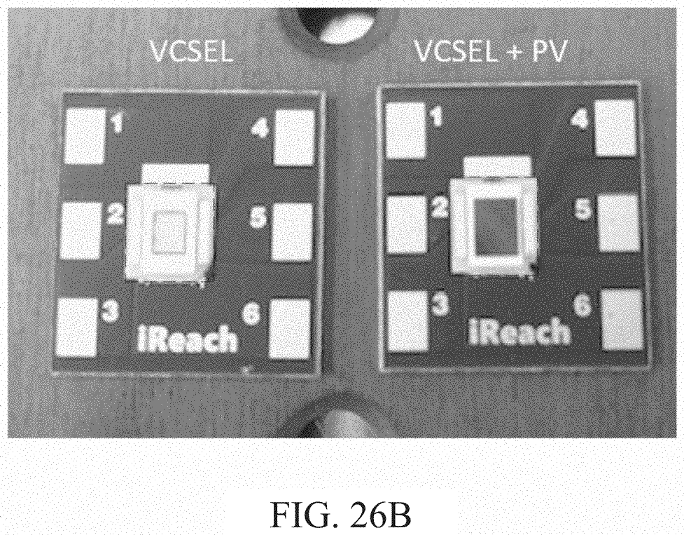

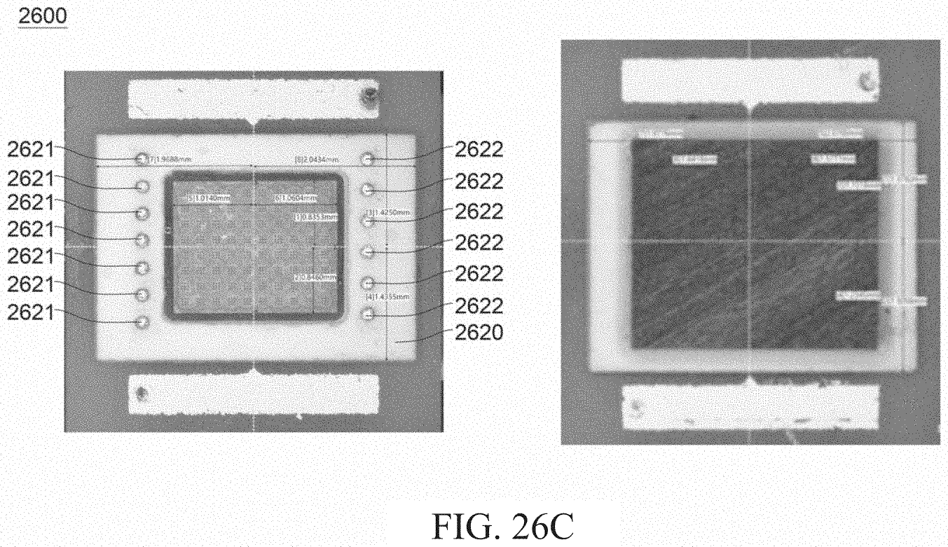

Please refer to FIG. 26A through FIG. 26C. FIG. 26A through FIG. 26C illustrate optical-microscopic photos showing top views of the chip package structure according to an exemplary embodiment. The outermost layer of the chip package structure shown in FIG. 26A comprises an opaque material. The left-hand-side of FIG. 26B illustrates a top view of the VCSEL chip connected to the substrate, and the right-hand-side of FIG. 26B illustrates the photovoltaic chip connected to the substrate through the housing and covering the VCSEL chip. FIG. 26C illustrates an enlarged partial view of the region enclosed by the dotted line in FIG. 26B.

As shown in FIG. 26C, in this embodiment, the housing 2620 of the chip package structure 2600 comprises a plurality of first conductive connection members 2621 and a plurality of second conductive connection members 2622. The first conductive connection members 2621 are arranged in a row and aligned with the first electrode portion (such as the first electrode portion 1471 shown in FIG. 15F) after the second chip (not shown in FIG. 26C) is connected to the housing 2620. The second conductive connection members 2622 are arranged in a row and aligned with the second electrode portion (such as the second electrode portion 1472 shown in FIG. 15F) after the second chip (not shown in FIG. 26C) is connected to the housing 2620. Moreover, the total number of the first conductive connection members 2621 is equal to the total number of the first conductive connection structures (such as the first conductive connection structures 143 shown in FIG. 1B) added with the total number of the first aligning connection structures (such as the first aligning connection structures 141 shown in FIG. 1B); the number of the first conductive connection members 2621 is also equal to the number of the first electrode connection structures (such as the first electrode connection structures 1471a1-1471a13 shown in FIG. 15F). The total number of the second conductive connection members 2622 is equal to the total number of the second conductive connection structures (such as the second conductive connection structures 144 shown in FIG. 1B) added with the total number of the second aligning connection structures (such as the second aligning connection structures 142 shown in FIG. 1B); the number of the second conductive connection members 2622 is also equal to the number of the second electrode connection structures (such as the second electrode connection structures 1472a1-1472a12 shown in FIG. 15F).

In the chip package structure according to one or some embodiments of the disclosure, the substrates of the first chip and the second chip may be chosen and designed based on their respective power levels and practical applications. Besides, electrical circuits may also be formed on the surfaces of the substrates or inside the substrates of the chip package structure, so that the substrate can be electrically connected to the chip set through the electrical circuits; that is, in one embodiment, materials suitable for laser drilling structure (LDS) technology, plastic covered with conductive material, ceramic substrate manufacturing process, printed circuit board manufacturing processes (PCB, FR4, BT, etc.) may be chosen as the substrate of the chip package structure.

As previously illustrated, in the chip package structure according to one or some embodiments of the disclosure, an upper chip serving as an emitting end and a lower chip serving as a receiving end are configured to be opposite to each other, so that the emitting end and the receiving end are packaged in the chip package structure to form a converter. In this converter, one of the chips (such as the first chip) of the chip set is adapted to be the emitting end to emit light, and the other chip (such as the second chip) of the chip set is adapted to be the receiving end to receive the emitted light to convert optical energy into electrical energy, and the series connection of the light receiving region of the second chip is then used to convert the electrical energy into a high voltage for output.

For example, the chip package structure according to one or some embodiments of the disclosure may be applied to an optical transformer (OT), wherein the series chip sets (such as a VCSEL chip paired with a photovoltaic chip) in the chip package structure are utilized to convert a low-voltage (such as less than 3 V) and large-current input into a high-voltage and large-current output for further applications. According to one or some embodiments, the optical transformer is especially applicable to miniature devices with high-voltage output, such as wearable automated external defibrillator (AED) devices.

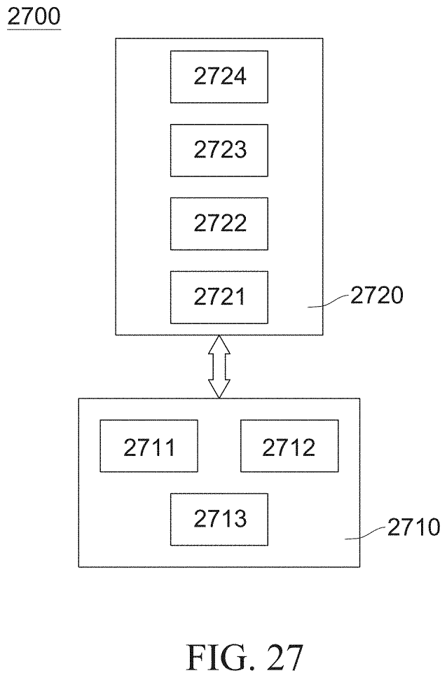

Please refer to FIG. 27. FIG. 27 illustrates a schematic diagram of an AED module according to an exemplary embodiment. As shown in FIG. 27, this exemplary embodiment is related to novel AED equipment 2700. The novel AED equipment 2700 comprises an AED device 2710 comprising any of the chip package structures illustrated in the previous exemplary embodiments and a hand-held device 2720 paired with the AED device 2710. The AED device 2170 comprises a power module 2711 for constant-voltage input current, a high-voltage power module 2712, and an electrode pad module 2713. The high-voltage power module 2712 comprises the chip package structure according to one or some embodiments of the disclosure as a transformer to provide the electrode pad module 2713 with a high voltage for defibrillation shock. In this exemplary embodiment, the AED device 2710 may be paired with a hand-held device (such as a smart phone) through a standard communication interface (such as universal serial bus, USB) to conduct information transmission. In this exemplary embodiment, the hand-held device 2720 comprises, beside its existing functional modules, a circuit module 2721, a power module 2722, a diagnosis module 2723, and a display module 2724. The circuit module 2721 is adapted to transmit signals among various modules, the power module 2722 provides power for various modules, and the diagnosis module 2723 is adapted to read the heart rate of the shock receiver before the AED device outputs a shock and then issue a signal corresponding to an advice based on the heart rate of the shock receiver, such as shock advised (such as when the heart rate of the shock receiver meets the standard of AED usage) or shock not advised (such as when the heartbeat of the shock receiver has stopped). The diagnosis module 2723 may even issue an audio instruction to the operator and display corresponding post-shock results (such as the heart rate of the shock receiver) using the display module 2724 to the operator.

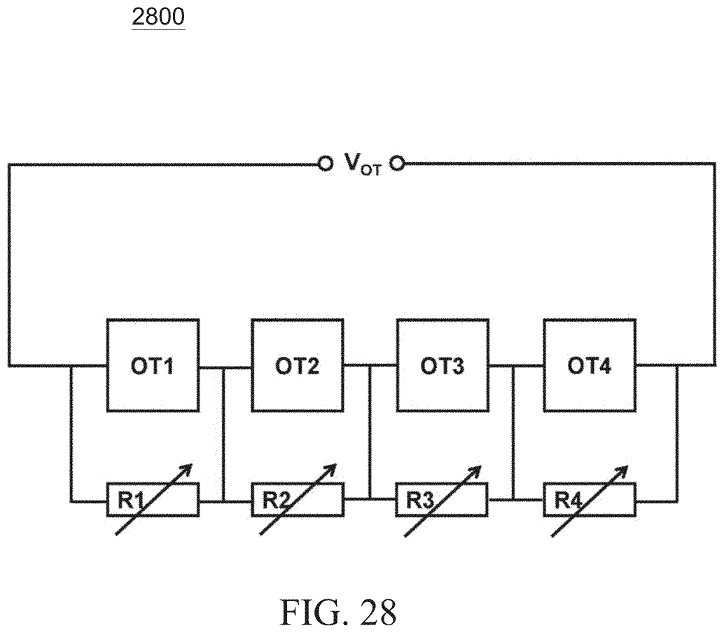

Please refer to FIG. 28. FIG. 28 illustrates a schematic block diagram of tunable optical transformer device according to an exemplary embodiment. The tunable optical transformer device 2800 comprises a plurality of optical transformers OT1, OT2, OT3, OT4 and a plurality of variable resistors R1, R2, R3, R4. As shown in FIG. 28, the electric output ends of the optical transformers OT1, OT2, OT3, OT4 are connected to one another in series, and the optical transformers OT1, OT2, OT3, OT4 are respectively electrically connected to the variable resistors R1, R2, R3, R4.

In this exemplary embodiment, the optical transformers OT1, OT2, OT3, OT4 each may be a converter comprising one or more of the chip packages structures 100, 200, 300, 400, 500, 600, 700, 800, 2200, 2300 according to the previous exemplary embodiments. The optical transformers OT1, OT2, OT3, OT4 may each be an independent converter. Alternatively, in some other exemplary embodiments, the optical transformers OT1, OT2, OT3, OT4 may be integrally packaged on the same base/substrate within an integrated converter.

Please refer to FIG. 28. In this exemplary embodiment, the optical transformers OT1, OT2, OT3, OT4 may be respectively configured to be converters having different output voltage specifications. For example, the output voltage specification of the optical transformer OT1 is 1 V, the output voltage specification of the optical transformer OT2 is 2 V, the output voltage specification of the optical transformer OT3 is 4 V, and the output voltage specification of the optical transformer OT4 is 8 V, but the instant disclosure is not limited thereto. In this exemplary embodiment, the optical transformer device 2800 further comprises a controller (not shown in the figures) coupled to the variable resistors R1, R2, R3, R4. The controller is adapted to control the resistances of the resistors R1, R2, R3, R4 to keep the optical transformer device 2800 from forming an open circuit during operation. Furthermore, the resistances of the resistors R1, R2, R3, R4 may be independently controlled, so that the output voltage V_{OT} of the optical transformer device 2800 can be modulated and outputted within the range of 0 V to 15 V, but the instant disclosure is not limited thereto.

Please continue to refer to FIG. 28. The number of the optical transformers OT in the optical transformer device 2800, the output voltage specifications of the optical transformers OT, and the number of the resistors are not limited to the embodiment shown in FIG. 28. Specifically, in this embodiment, the specification of the output voltage V_{OT} of the optical transformer device 2800 may be designed by configuring the number of the optical transformers OT or different output voltage specifications of the optical transformers OT to realize optical transformer devices with low output voltage specification or high voltage specification, or optical transformer devices with tunable output voltage ranges. For example, in an exemplary embodiment, the optical transformer device 2800 may comprise five optical transformers OT1 with 1V output voltage specification, and thus the output voltage V_{OT} of the optical transformer device 2800 can be modulated and outputted within the range of 0 V to 5 V, but the instant disclosure is not limited thereto.

The controller may be coupled to the resistors R1, R2, R3, R4 through a direct contact connection, a wired connection, or wirelessly. The controller may be integrated into the structure of the optical transformer device 2800, or the controller may be a remote controller, but the instant disclosure is not limited thereto. The controller may be realized using analog circuit structure, digital circuit structure, or integrated analog-digital circuit structure, or the controller may be realized using software program, firmware, or integrated soft-hardware, but the instant disclosure is not limited thereto. The controller may comprise a memory and a processor, wherein the memory may be a non-volatile memory (such as a flash memory, a phase-change memory (PRAM), a magnetoresistive RAM (MRAM), a resistive RAM (ReRAM), or a ferroelectric RAM (FRAM)), a volatile memory (such as a static RAM (SRAM), a dynamic RAM (DRAM), a synchronous DRAM (SDRAM), or the like). Also, the processor may be a central processing unit (CPU), a graphics processing unit (GPU), a microcontroller unit (MCU), or an application-specific integrated circuit (ASIC), so that the processor is able to access the programs and commands stored in the memory and perform corresponding control functions, but the instant disclosure is not limited thereto.

As previously illustrated, using the concept according to one or some embodiments of the disclosure, miniaturized devices with high-voltage or large-current output can be realized to conform to application requirements in industries of medical care, vehicle, wearable device, novel electronic device, and so forth. For example, utilizing an AED device having the chip package structure disclosed in one or some embodiments of this instant disclosure, a novel AED device which is miniaturized, portable, and smart can be realized with large application potentials.

What is claimed is:

1. A chip package structure comprising:
a substrate comprising a first surface and a second surface being opposite surfaces of the substrate;
a chip set disposed in a chip region on the first surface and electrically connected to the substrate, wherein the chip set comprises a first chip and a second chip, and an active surface of the second chip faces an active surface of the first chip.

2. The chip package structure according to claim 1, further comprising a heat dissipation layer, wherein the heat dissipation layer directly contacts either or both of the first chip and the second chip.

3. The chip package structure according to claim 1, wherein the first chip is a light-emitting chip, the active surface of the first chip is a light-emitting surface, the second chip is a light-receiving chip, and the active surface of the second chip is a light-receiving surface.

4. The chip package structure according to claim 3, wherein a distance between the light-emitting surface and the light-receiving surface is in a range between 1 μm and 30 μm.

5. The chip package structure according to claim 1, further comprising a plurality of first electrical conduction posts and a plurality of second electrical conduction posts, wherein the first electrical conduction posts and the second electrical conduction posts penetrate the substrate and extend to the second surface, the first chip is electrically connected to the substrate through the first electrical conduction posts, and the second chip is electrically connected to the substrate through the second electrical conduction posts.

6. The chip package structure according to claim 5, further comprising a middle layer between the first chip and the second chip, wherein the middle layer is an electrical insulation layer, a light-transmissive layer, or an electrically insulating light-transmissive layer.

7. The chip package structure according to claim 1, further comprising: a housing disposed on the first surface of the substrate and enclosing the chip region, wherein the first chip is on the first surface, the chip package structure further comprises a plurality of electrical conduction structures, the electrical conduction structures penetrate the housing, the housing has a height greater than a thickness of the first chip, and the second chip is on the housing and is electrically connected to the substrate through the electrical conduction structures.

8. The chip package structure according to claim 7, further comprising a plurality of conductive connection structures on the second chip, wherein the conductive connection structures are connected to the electrical conduction structures, and the second chip is electrically connected to the substrate through the conductive connection structures and the electrical conduction structures.

9. The chip package structure according to claim 8, further comprising a plurality of first aligning connection structures and a plurality of second aligning connection structures, wherein the first aligning connection structures and the second aligning connection structures are on the second chip and aligned with the conductive connection structures, the first aligning connection structures and the second aligning connection structures are adapted for alignment of the second chip in the chip package structure, and a number of the first aligning connection structures is different from a number of the second aligning connection structures.

10. The chip package structure according to claim 9, wherein no electricity conduction is formed between the first aligning connection structures and the second aligning connection structures.

11. The chip package structure according to claim 8, further comprising a plurality of aligning connection structures on the second chip and aligned with the conductive connection structures, wherein the aligning connection structures are adapted for alignment of the second chip in the chip package structure.

12. The chip package structure according to claim 1, wherein the first chip comprises a plurality of blocks which are arranged orderly, at least two of the blocks comprise a plurality of columnar structures, and a number of the columnar structures in one of the at least two blocks is identical to a number of the columnar structures in any other one of the at least two blocks.

13. The chip package structure according to claim 12, wherein the second chip comprises a plurality of mesa structures which are arranged orderly, a groove separates any two neighboring ones of the mesa structures, and each of the mesa structures of the second chip is aligned with a corresponding one of the blocks of the first chip.

14. The chip package structure according to claim 13, wherein a first distance is a shortest distance between the columnar structures of two neighboring ones of the blocks of the first chip, the groove of the second chip has a first width, and the first distance is greater than the first width.

15. The chip package structure according to claim 14, wherein the first chip comprises a third surface and a fourth surface, the third surface faces the first surface, the fourth surface faces away the first surface, and the third surface and the fourth surface are opposite surfaces of the first chip; the second chip comprises a fifth surface and a sixth surface, the fifth surface faces the fourth surface, the sixth surface faces away the fourth surface, and the fifth surface and the sixth surface are opposite surfaces of the second chip; the fourth surface is the active surface of the first chip, and the fifth surface is the active surface of the second chip;
wherein the chip package structure further comprises:
a first conduction structure and a second conduction structure, wherein the first conduction structure is on the third surface of the first chip and connected to one of the first electrical conduction posts, and the second conduction structure is on the third surface of the first chip and connected to another one of the first electrical conduction posts, so that the first chip is electrically connected to the first electrical conduction posts; and
a first conductive connection structure and a second conductive connection structure, wherein the first conductive connection structure is on the sixth surface and connected to one of the second electrical conduction posts through a wire, and the second conductive connection structure is on the sixth surface and connected to another one of the second electrical conduction posts through another wire, so that the second chip is electrically connected to the second electrical conduction posts.

16. The chip package structure according to claim 14, wherein the first chip comprises a third surface and a fourth surface, the third surface faces the first surface, the fourth surface faces away the first surface, and the third surface and the fourth surface are opposite surfaces of the first chip; the second chip comprises a fifth surface and a sixth surface, the fifth surface faces the fourth surface, the sixth surface faces away the fourth surface, and the fifth surface and the sixth surface are opposite surfaces of the second chip; the fourth surface is the active surface of the first chip, and the fifth surface is the active surface of the second chip;
wherein the chip package structure further comprises:
a first conduction structure and a second conduction structure, wherein the first conduction structure is on the third surface of the first chip and the second conduction structure is on the fourth surface of the first chip, the first conduction structure is connected to at least one of the first electrical conduction posts, and the second conduction structure is connected to at least another one of the first electrical conduction posts through a first wire, so that the first chip is electrically connected to the substrate; and
a first conductive connection structure and a second conductive connection structure, wherein the first conductive connection structure is on the sixth surface and connected to one of the second electrical conduction posts through a second wire, and the second conductive connection structure is on the sixth surface and connected to another one of the second electrical conduction posts through another second wire, so that the second chip is electrically connected to the substrate.

17. The chip package structure according to claim 12, wherein in the at least two blocks, a distance between any two neighboring ones of the columnar structures is identical to a distance between any other two neighboring ones of the columnar structures.

18. The chip package structure according to claim 12, wherein control of the columnar structures in one of the blocks is independent of control of the columnar structures in another one of the blocks.

19. The chip package structure according to claim 12, wherein the first chip has a first geometric center, the second chip has a second geometric center, and the first geometric center and the second geometric center are substantially aligned with each other.

20. An automated external defibrillator comprising:
a power module adapted to provide a constant-voltage input current;
a high-voltage power module comprising the chip package structure according to any claim from claims 1 and adapted to receive the constant-voltage input current and output a high-voltage current; and
an electrode pad module coupled to the high-voltage power module and adapted to output a high voltage so as to deliver a defibrillation shock.
